(19) United States
(12) Patent Application Publication
(10) Pub. No.: US 2021/0154281 A1
(43) Pub. Date: May 27, 2021

(54) CELL-BASED CANCER VACCINES AND CANCER THERAPIES

(71) Applicant: Massachusetts Institute of Technology, Cambridge, MA (US)

(72) Inventors: Darrell Irvine, Arlington, MA (US); Michael Yaffe, West Roxbury, MA (US); Ganapathy Sriram, Quincy, MA (US); Lauren Milling, Cambridge, MA (US)

(21) Appl. No.: 17/033,050

(22) Filed: Sep. 25, 2020

Related U.S. Application Data
(60) Provisional application No. 62/940,808, filed on Nov. 26, 2019.

(51) Int. Cl. A61K 39/00; A61K 35/15; A61K 35/17; A61K 45/06; G01N 33/50

(52) U.S. Cl. CPC A61K 39/0011; A61K 35/15; A61K 35/17; A61K 2039/54; G01N 33/5011; A61K 2039/5152; A61K 45/06

(57) ABSTRACT

Described are cell-based cancer vaccines and anti-cancer immunotherapies. The vaccines include isolated tumor cells activated with one or more genotoxic drugs, and, optionally, treated with one or more MK2 inhibitors. The activated cells are highly immunogenic non-proliferative cells, and may be tested for immunogenicity ex vivo for priming T cells by co-incubating the isolated activated cells with dendritic cells and T cells. The vaccines are typically administered into patient's tumor to provide an intratumoral immune activation. Immune checkpoint inhibitor(s) (ICI) may be administered before, during, or after vaccine administration. ICI may be a component of the vaccine. The vaccines confer heightened cytotoxic immune response against the cancer cells, induce tumor regression, and enhance survival from cancer. The vaccines prevent tumor recurrence and induce a long-lasting anti-tumor immunological memory.

### CROSS REFERENCE TO RELATED APPLICATIONS

This application claims the benefit of and priority to U.S. Provisional Application No. 62/940,808, filed Nov. 26, 2019, and is hereby incorporated herein by reference in its entirety.

### STATEMENT REGARDING FEDERALLY SPONSORED RESEARCH

This invention was made with Government support under Grant Nos. R01 ES015339 and R35 ES028374 awarded by the National Institutes of Health (NIH). The Government has certain rights in this invention.

### REFERENCE TO SEQUENCE LISTING

The Sequence Listing submitted as a text file named “MIT_21498_ST25.txt,” created on Sep. 23, 2020, and having a size of 542 bytes is hereby incorporated by reference pursuant to 37 C.F.R § 1.52(e)(5).

### FIELD OF THE INVENTION

The invention is generally directed to cell-based cancer vaccines and immune therapies against cancer.

### BACKGROUND OF THE INVENTION

Therapeutic manipulation of the immune system as a component of anti-cancer therapy has seen major advances over the last decade with the development of immune checkpoint inhibitors (ICI) targeting the PD-1/PD-L1 and CTLA-4 axes (Ribas and Wolchok, Science, 359(6382):1350-1355 (2018)). Certain tumor types show impressive clinical responses to these agents, particularly melanoma (Larkin et al., N Engl J Med., 373:23-34 (2015)), non-small cell lung cancer (Borghaei et al., N Engl J Med., 373:1627-1639 (2015), Brahmer et al., N Engl J Med., 373:123-135 (2015), Reck et al., N Engl J Med., 375:1823-1833 (2016)), and microsatellite instability (MSI)-high colon cancer (Overman et al., J Clin Oncol., 36:4_suppl, 554-554 (2018), Overman et al., Lancet Oncol., 18(9):1182-1191 (2017)). However, the majority of patients with most common tumor types, including breast cancer (Adams et al., Ann Oncol., 30(3):397-404 (2019), Adams et al., Ann Oncol., 30(3):405-411 (2019)), ovarian cancer (Nivolumab With or Without Ipilimumab in Treating Patients With Recurrent or High Grade Gynecologic Cancer With Metastatic Peritoneal Carcinomatosis (ClinicalTrials website, Identifier: NCT03508570), Pietzner et al., Journal of Gynecologic Oncology., 29(6):e93 (2018)), and microsatellite-stable (MSS) colon cancer (Eng et al., Lancet Oncol. 20(6):849-861 (2019)) show much lower response rates, and it has been estimated that the overall percentage of all cancer patients who will respond to immune checkpoint inhibitors alone is less than 13% (Haslam and Prasad, JAMA Netw Open. 2(5):e192535 (2019)). Identifying mechanisms that would enhance these response rates and prolong the durability of the response remains an unmet clinical need.

Conventional DNA-damaging chemotherapy with the DNA topoisomerase I and II inhibitors doxorubicin, etoposide, camptothecin and irinotecan, the platinum agents cisplatin and oxaliplatin, and the alkylating agent cyclophosphamide remain a mainstay of clinical cancer treatment. A combination of cisplatin with anti-PD1 was recently approved as a first line treatment for NSCLC in patients with >50% PD-L1 expression in tumor cells (Gandhi et al., N Engl J Med., 24(8):1178-1191 (2018), Langer et al., Lancet Oncol., 17(11):1497-150 (2016)). However, the rationale for this strategy did not take into account the lack of ability of cisplatin treatment to enhance tumor immunogenicity (Martins et al., Oncogene, 30(10):1147-58 (2011)).

Some, but not all, DNA-damaging chemotherapeutic agents have been shown to stimulate the release of danger signals which could potentially enhance dendritic cell processing and presentation of tumor antigens (Obeid et al., Nat Med., 13(1):54-61 (2007)). Nonetheless, how to best combine chemotherapy with ICI for different tumor types is still not clear.

Two approaches that could potentially enhance the response of tumors to immunooncology therapies are the use of tumor cell vaccines, or the combination of chemotherapeutic drugs with immune checkpoint inhibitors. Examples of vaccination strategies designed to target the immune response to tumor-specific antigens have included identifying cancer-specific mutations by whole exome sequencing of tumor biopsies followed by vaccinating with a mixture of cancer specific mutant peptides or mRNA (Ott et al., Nature, 547(7662):217-22 (2017), Sahin et al., Nature, 547(7662):222-226 (2017)), or vaccinating with autologous irradiated tumor cells in combination with cell lines engineered to express GM-CSF (Curry et al., Clin Cancer Res., 22(12):2885-96 (2016)), or allogeneic irradiated tumor cells expressing GM-CSF (Dranoff et al., Proc Nall Acad Sci USA., 90(8):3539-43 (1993), Lipson et al., J Transl Med., 13:214 (2015)). The former approach requires extensive sequencing and computational analysis, followed by rapid synthesis of a patient-specific vaccine, which is both time consuming and expensive. The latter approach, which involves intradermal injection of allogeneic engineered tumor cells is well tolerated in patients, however, it has not been successful in clinical trials so far (GVAX® Vaccine for Prostate Cancer vs Docetaxel & Prednisone in Patients With Metastatic Hormone-Refractory Prostate Cancer (ClinicalTrials website, Identifier: NCT00089856), Docetaxel in Combination With GVAX® Immunotherapy Versus Docetaxel and Prednisone in Prostate Cancer Patients (ClinicalTrials website, Identifier: NCT00133224)).

Importantly, neither of these vaccination strategies directly access intra-tumoral stimulatory dendritic cells (DCs) or DCs in the tumor-draining lymph node, which may be important in obtaining strong T-cell responses against the tumor. A subset of intra-tumoral dendritic cells, characterized by their surface expression of CD103 in mice and BDCA-3 in humans, has been identified as having unique capabilities of cross-presenting tumor-associated antigens to CD8+ T-cells and recruiting T-cells to the tumor microenvironment through CXCL9/10 (Hildner K., Science, 322(5904):1097-100 (2008), Spranger, et al., Cancer Cell, 8; 31(5):711-723.e4. doi: 10.1016/j.cce11.2017.04.003 (2017), Roberts, et al., Cancer Cell, 30(2):324-336. doi: 10.1016/j.ccell.2016.06.003 (2016)). The levels of these DCs in the tumor microenvironment was shown to correlate with better overall survival in melanoma patients receiving immune checkpoint inhibitors (Barry, et al., Nature Medicine, 24:1178-1191 (2018)), consistent with the importance of these cells in enhancing anti-tumor immune responses.

For many tumor types, immunotherapy has been reserved as a second- or third-line treatment option in patients who have failed prior treatment with cytotoxic agents (FDA approvals Hematology/Oncology (Cancer) Approvals & Safety Notifications, FDA website). However, early combination of chemotherapy with immune checkpoint inhibitors as a first line therapeutic modality was approved for EGFR, ALK, and ROS negative non-small cell lung cancer (NSCLC) using cisplatin and pembrolizumab (Gandhi, et al., N Engl J Med., 378:2078-2092 (2018)), and for head and neck squamous cell carcinomas (HNSCC) using platinum agents, 5-FU, and pembrolizumab (Burtness et al., Lancet., 394(10212):1915-1928 (2019)).

Data supporting this approach comes from the KEYNOTE-189 trial, which showed a median progression-free survival of 8.8 months in patients with NSCLC that were treated with a combination of cisplatin or carboplatin, pemetrexed, and pembrolizumab, compared to 4.9 months in patients who were treated with chemotherapy alone. However, over 65% of the patients who received this chemotherapy and immunotherapy combination continued to have progressive disease (Gandhi, et al., N Engl J Med., 378:2078-2092 (2018)). Similarly, the KEYNOTE-048 trial, performed in patients with recurrent unresectable HNSCC in which the tumor contained greater than 1% of cells staining positively for PD-L1 failed to show any improvement in progression-free survival in patients treated with cisplatin or carboplatin, 5-FU, and pembrolizumab, compared to those treated with the same chemotherapy plus cetuximab, although there was an increase in median overall survival from 10.7 months to 13 months when pembrolizumab was included in the combination (Burtness et al., Lancet., 394(10212):1915-1928 (2019)).

Thus, there remains a need for identifying mechanisms that would enhance response rates to the combination of immune checkpoint blockade and chemotherapy, and prolong the durability of the response, and improved anti-cancer immunotherapies that reduce tumor burden and preferably provide a long-lasting anti-tumor immunological memory.

Therefore, it is the object of the present invention to provide anti-cancer vaccines that reduce tumor burden and preferably provide a long-lasting anti-tumor immunological memory.

It is another object of the present invention to provide methods of making the anti-cancer vaccines.

It is yet another object of the present invention to provide methods of using the anti-cancer vaccines.

### SUMMARY OF THE INVENTION

Described are cell-based cancer vaccines and anti-cancer immunotherapies. The vaccines typically include isolated tumor cells, isolated from a patient's tumor, and activated with one or more genotoxic drug(s). The isolate activated tumor cells are typically used as live, injured cells, but not dead cells. The cell may also be treated with a mitogen-activated protein kinase-activated protein kinase 2 (MK2) inhibitor. The isolated and activated cells are typically non-proliferative cells with DNA damage, growth arrest, and/or necroptosis, and have an increased immunogenic potential. The vaccines may include one or more immune checkpoint inhibitors (ICI). The vaccines may also include autologous or allogeneic antigen presenting cells (APCs), T cells, or a combination thereof.

The isolated activated tumor cells of the vaccine may have immunogenic cell death markers, such as increased calreticulin exposure on cell surface and activated receptor-interacting protein kinase 1 (RIPK1) and/or activated NF-κB signaling, and/or markers of intact or increased stress signaling, including DNA damage signaling, such as substrates of ATM and/or ATR, phosphorylated p38MAPK, or a combination thereof. In some embodiments, the cells may have increased activation and/or not substantially reduced activation of NF-κB signaling (e.g., compared to unactivated cells). In some embodiments, NF-κB signaling is not artificially inhibited with a further compound (e.g., NF-κB inhibitor) that inhibits NF-κB signaling. The isolated activated tumor cells typically have an increased immunogenic potential. For example, the cells may induce an increase in the percentage of interferon (IFN)-gamma-producing cytotoxic T cells when the activated cells are co-cultured with dendritic cells and T-cells as compared to the percentage of interferon (IFN)-gamma-producing cytotoxic T cells when isolated control cells (not activated with genotoxic drug(s)) are co-cultured with dendritic cells and T-cells.

The vaccines are useful for treating a patient with cancer, and/or preventing recurrence of the cancer. The vaccines are typically administered into the patient's tumor to provide an intratumoral immune activation. Immune checkpoint inhibitor(s) (ICI) may be administered before, during, or after vaccine administration. The vaccines typically confer a heightened cytotoxic immune response against the cancer cells, induce tumor regression, enhance survival from cancer, or a combination thereof. Preferably, the vaccines can prevent tumor recurrence and induce a long-lasting anti-tumor immunological memory.

Also described are personalized treatments of patients with cancer. The treatments typically include administering into a tumor of the patient an effective amount of the patient's own activated tumor cells having an increased immunogenic potential.

Also described are assays for testing genotoxic drug(s) to identify drug(s) and dosages/concentrations thereof that produce activated tumor cells with increased immunogenic potential. The drug and concentration thereof is typically one that injures the cell, with being a concentration that induces maximal cell death. The assay typically includes isolating tumor cells from the patient's tumor, culturing samples of the isolated tumor cells with genotoxic drugs to produce activated cells, and testing the activated cells for the presence of immunogenic cell death markers. The assay may additionally or alternatively include testing the activated cells for the potential to induce an increased percentage of interferon (IFN)-gamma-producing cytotoxic T cells when the activated cells are co-cultured with dendritic cells and T-cells.

### BRIEF DESCRIPTION OF THE DRAWINGS

FIG. 1A is a diagram of the in vitro experimental system with sequential co-cultures of chemotherapy drug-treated B16-Ova or MC-38-Ova cells, primary bone marrow-derived dendritic cells (BMDC) and OT-1 CD8+ T-cells for evaluating BMDC-mediated T-cell priming FIG. 1B is a bar graph showing quantification of IFNg+CD8+ T-cells (% CD3+CD8+IFNγ+ T cells) from 5 independent experiments. The first lane (−) indicates the percentage of IFNg+CD8+ T-cells produced by co-culture of BMDCs and T-cells in the absence of B16-Ova cells. * indicates p<0.0001 when compared to DMSO-treated control cells using ANOVA followed by Dunnett's multiple comparisons test. FIG. 1C is a bar graph showing quantification of BMDC-mediated induction of IFN-γ+ CD8+ T-cells by chemotherapy-treated B16-Ova cells. The first lane (−) indicates the percentage of IFNg+CD8+ T-cells produced by co-culture of BMDCs and T-cells in the absence of any B16-Ova cells. * indicates p<0.006 when compared to (−) sample using ANOVA followed by Dunnett's multiple comparisons test. FIG. 1D is a bar graph showing quantification of BMDC-mediated induction of IFN-γ+ CD8+ T-cells by chemotherapy-treated MC-38-Ova cells from 3 independent experiments. The first lane (−) indicates the percentage of IFNg+CD8+ T-cells produced by co-culture of BMDCs and T-cells in the absence of MC-38-Ova cells. * indicates p<0.0001 when compared to DMSO-treated control using ANOVA followed by Dunnett's multiple comparisons test. In all panels, error bars represent SEM. FIG. 1E is a bar graph showing AnnexinV/DAPI staining 48 hours after treatment with the indicated drugs and concentrations. Dox—doxorubucin, Etop—etoposide, Mito—mitoxantrone, Cis—cisplatin, Oxal—oxaliplatin, 5-FU—5-fluorouracil, Pac—paclitaxel, Iri—irinotecan, CPT—camptothecin, CPM—cyclophosphamide Error bars represent range obtained from at least two independent experiments. FIGS. 1F and 1G are bar graphs showing AnnV/DAPI staining as analyzed by flow cytometry of the total (all), attached, or floating (suspension) fractions of B16-Ova cells after treatment with Etoposide (50 uM) (in 1F) or Mitoxantrone (10 uM) (in 1G) for 24 hours. Quantification of live cells (AnnV and DAPI double negative; black bars) and dead cells (AnnV or DAPI single or double positive; gray bars) in each fraction from three independent experiments is shown. Errors represent SEM. FIGS. 1H and 1I are bar graphs showing AnnV/DAPI staining as analyzed by flow cytometry of the total (all), attached, or floating (suspension) fractions of MC-38-Ova cells after treatment with Etoposide (50 uM) (in 1H) or Mitoxantrone (10 uM) (in 1I) for 24 hours. Quantification of live cells (AnnV and DAPI double negative; black bars) and dead cells (AnnV or DAPI single or double positive; gray bars) in each fraction from three independent experiments is shown. Errors represent SEM. FIG. 1J is a bar graph showing quantification (from three independent experiments) of IFN-γ+ CD8+ T-cells induced by co-culture of BMDC with B16-Ova cells treated with etoposide from 0 to 100 uM for 24 h. The first lane (−) indicates the percentage of IFN-γ+ CD8+ T-cells produced by co-culture of BMDCs and T-cells in the absence of B16-Ova cells. Error bars indicate SEM. * indicates p<0.03 using ANOVA followed by Sidak's multiple comparisons test. FIG. 1K is a bar graph showing quantification (from three independent experiments) of IFN-γ+ CD8+ T-cells induced by co-culture of BMDC with B16-Ova cells treated with mitoxantrone from 0 to 100 uM for 24 h. The first lane (−) indicates the percentage of IFN-γ+ CD8+ T-cells produced by co-culture of BMDCs and T-cells in the absence of B16-Ova cells. Error bars indicate SEM. * indicates p<0.0001 using ANOVA followed by Sidak's multiple comparisons test. FIGS. 1L-1M show quantification (from two to three independent experiments) of the proportion of live (AnnV and DAPI double negative; black bars) and dead (sum total of AnnV and/or DAPI single or double positive; grey bars) cells after treatment of B16-Ova cells for 24 h with etoposide or mitoxantrone as indicated. Error bars indicate SEM. FIG. 1N-1O are bar graphs showing quantification (from three independent experiments) of IFN-γ+ CD8+ T-cells induced by co-culture of BMDC with the indicated B16-Ova cell fractions obtained after treatment with etoposide or mitoxantrone. B16-Ova cells were treated with etoposide at 50 uM or mitoxantrone at 10 uM and fractionated into live cells (AnnV and DAPI double negative) and dead cells (AnnV and/or DAPI single or double positive) as described in Methods. Lysate and cell-free supernatants were also obtained as described. BMDC was co-cultured with each of the following fractions or combinations of fractions for 24 h before OT-1 CD8+ T-cells were added: (Live+dead) refers to the whole treated cell mixture, (Live) refers to the live cell fraction, (Dead) refers to the dead cell fraction, Sup refers to Cell-free supernatant, (Dead+Sup) refers Dead cells combined with cell-free supernatant, (Dead) refers to the dead cells without cell-free supernatant. Error bars indicate SEM. * indicates p<0.0001 using ANOVA followed by Dunnett's multiple comparisons test. FIGS. 1P and 1Q are bar graphs showing quantification (from three independent experiments) of IFN-γ+ CD8+ T-cells induced by co-culture of BMDC with the indicated MC-38-Ova cell fractions obtained after treatment with etoposide or mitoxantrone as described in 1N and 10 and in Methods. Error bars indicate SEM. * indicates p<0.0003 using ANOVA followed by Dunnett's multiple comparisons test.

FIG. 2A is a bar graph showing the percentage of B16-Ova tumor cells (% CALR+cells) displaying surface calreticulin 24 hours after the indicated treatment from a representative experiment. FIGS. 2B and 2C are bar graphs showing levels of HMGB1 (ng/ml) (FIG. 2B) and ATP (nM) (FIG. 2C) in the culture media measured 24-48 hours after the indicated treatment. Results are from 4 independent experiments, with error bars indicating SEM. Data in FIG. 2B was analyzed by comparison to DMSO-treated controls using ANOVA followed by Dunnett's multiple comparisons test. * indicates p<0.03. FIG. 2D is a bar graph showing quantification of IFNg+CD8+ T-cells. Results represent 3 independent experiments with error bars indicating SEM. Data were analyzed by comparison of drug-treated calreticulin knock-down cells (siCalR) to their respective drug-treated control knockdown cells (siCtrl) using a two-tailed t-test. * indicates p<0.002. FIG. 2E is a bar graph showing quantification of IFNg+CD8+ T-cells induced by BMDC following incubation with etoposide- or mitoxantrone-treated B16-Ova cells that were co-treated with the indicated DNA damaging agent plus either Necrostatin-1 (Nec-1) or Z-VAD. First lane (−) defined as in FIG. 1B. Results represent 3 independent experiments with error bars indicating SEM. * indicates p<0.005 Z-VAD or Nec-1 treated cells were compared with their untreated etoposide controls using a 2-tailed t-test with Bonferroni correction.

FIG. 3A is a diagram of the experimental design and dosing regimen used for testing intra-tumoral administration of etoposide in the presence or absence of systemic anti-PD1 and anti-CTLA4. FIGS. 3B-3E are graphs showing tumor growth curves in mice bearing B16-Ova tumors treated with intra-tumoral saline (Saline IT; FIG. 3B) or etoposide (Etop IT; FIG. 3C) alone, or intra-tumoral saline (FIG. 3D) or etoposide (FIG. 3E) in the presence of systemic anti-PD1 and anti-CTLA4. The number of mice in each group is indicated. One mouse in the Etop IT+anti-PD1/CTLA4 group did not show tumor growth beyond 4 mm² throughout the experiment and was excluded. FIG. 3F is a graph showing Kaplan-Meier survival curves of the mice in this experiment described in FIGS. 3B-3E. Survival of the Etop IT+anti-PD1/CTLA4 treatment group was not significantly different from that of the Saline IT+anti-PD1/CTLA4 group (log-rank test). FIG. 3G is a graph showing quantification of IFNg+CD8+ T-cells. Error bars represent SEM. * indicates p<0.0001, p<0.0005, p<0.002 respectively for DC number dilutions 1×, 0.5× and 0.25× compared to their respective negative (−) controls (one-tailed T-test with Bonferroni correction). FIG. 3H is a graph showing quantification of IFNg+CD8+ T-cells induced by BMDC after co-culture with etoposide-treated B16-Ova cells when both BMDC and T-cells were exposed to etoposide compared to when only B16-Ova cells were exposed. Error bars represent SEM. * indicates p<0.0001 (one-tailed t-test).

FIG. 4A is a diagram of the experimental design and dosing regimen used for testing intra-tumoral administration of etoposide-treated B16-Ova cells (tumor cell vaccine) in the presence or absence of systemic anti-PD1 and anti-CTLA4. FIGS. 4B-4E are graphs showing tumor growth curves for mice treated with intra-tumoral saline alone (Saline IT; FIG. 4B) or ex vivo etoposide-treated B16-Ova cells alone (Tumor cell vaccine IT; FIG. 4C), or intra-tumoral saline (FIG. 4D) or ex vivo etoposide-treated B16-Ova cells (FIG. 4E) in the presence of systemic anti-PD1 and anti-CTLA4. ‘n’ indicates the number of mice in each group. FIG. 4F is a graph showing Kaplan-Meier survival curves of this experiment described in FIGS. 4B-4E. * indicates p<0.02 when compared to the group treated with Saline IT+anti-PD1/CTLA4 (log-rank test). FIG. 4G is a graph showing the average tumor cross-sectional area on Day 21 for each treatment group. Error bars indicate SEM. * indicates p<0.02 when compared to the group treated with Saline IT+anti-PD1/CTLA4 (one-tailed t-test). FIG. 4H is a graph showing frequency of circulating H2-Kb/SIINFEKL (SEQ ID NO:1)-specific CD8+ T-cells from mice following the indicated treatments. Treatment groups shown in FIG. 3G are also included for comparison. * indicates p<0.04 (one-tailed t-test). FIG. 4I is a graph showing tumor growth curves in 5 naive mice and 5 mice that demonstrated complete tumor regression following the tumor cell vaccine+systemic anti-PD1/CTLA4 were re-challenged in the opposite flank with 100,000 live B16-Ova cells. Error bars indicate SEM.

FIG. 5A is a diagram of the experimental design and dosing regimen used to test the effect of intra-tumoral etoposide-treated B16-Ova cells in combination with systemic anti-PD1/CTLA4, on the frequency of intra-tumoral DC. FIG. 5B is a bar graph showing quantification of intra-tumoral CD11b-CD103+DC1 and CD11b+CD103-DC2 subsets from treated tumors analyzed by flow cytometry. Error bars represent SEM. * indicates p<0.04 (one-tailed t-test). FIGS. 5C-5E are graphs showing tumor growth curves of Batf3 KO mice treated with intra-tumoral saline (FIG. 5C), intra-tumoral saline in combination with systemic anti-PD1 and anti-CTLA4 antibodies (FIG. 5D), or etoposide-treated B16-Ova cells (tumor cell vaccine) in combination with systemic anti-PD1 and anti-CTLA4 antibodies (FIG. 5E). ‘n’ indicates the number of mice in each group. FIG. 5F is a graph of Kaplan-Meier survival curves of the experiment described in FIGS. 5C-5E. The survival curves are not significantly different (log-rank test, p=0.5220). FIG. 5G is a graph showing the frequency of circulating H2-Kb/SIINFEKL (SEQ ID NO:1)-specific CD8+ T-cells from WT and BATF3 (−/−) mice treated with the conditions indicated.

FIG. 6 is a bar graph showing the quantification of IFN-γ+ CD8+ T-cells (% CD3+CD8+IFN-γ+ T cells) induced by BMDC following incubation with etoposide-treated B16-Ova cells that were co-treated with either Bay 11-7085 (NF-κB inhibitor) or PF-3644022 (MK2 inhibitor). The first lane (−) indicates the percentage of IFN-γ+ CD8+ T-cells produced by co-culture of BMDCs and T-cells in the absence of B16-Ova cells. Error bars indicate SEM. * indicates p<0.0001 when compared to cells treated with Etoposide (50 uM) alone using ANOVA followed by Dunnett's multiple comparisons test.

FIG. 7A is a schematic of the experimental design to compare tumor infiltration of SIINFEKL (SEQ ID NO:1)-specific T-cells induced by the live injured cell fraction versus the dead cell fraction from the etoposide-treated B16-Ova cell mixture. FIG. 7B is a bar graph showing quantification of H2-Kb-SIINFEKL (SEQ ID NO:1)-specific CD8+ T-cells per mg of tumor in the groups in indicated.

FIGS. 8A and 8B are images showing live cell fractions from specific chemotherapy-treated B16-Ova cell mixtures analyzed by western blotting for serine-phosphorylated substrates of ATM and ATR (FIG. 8A) and also for phospho- and total p38MAPK as well as phospho (T334)- and total MK2 (FIG. 8B). FIG. 8C is a bar graph showing quantification of IFN-γ+ CD8+ T-cells induced by BMDC following incubation with etoposide-treated B16-Ova cells that were co-treated with either KU-55933 (ATM inhibitor), AZD6738 (ATR inhibitor) or NU7441 (DNA-PK inhibitor). The first lane (−) indicates the percentage of IFN-γ+ CD8+ T-cells produced by co-culture of BMDCs and T-cells in the absence of B16-Ova cells. Error bars indicate SEM. * indicates p<0.0001 when compared to cells treated with Etoposide (50 uM) alone using ANOVA followed by Dunnett's multiple comparisons test.

FIG. 9 is a bar graph showing quantification of IFN-γ+ CD8+ T-cells induced by BMDC following incubation with doxorubicin-treated B16-Ova cells at the doses indicated. The first lane (−) indicates the percentage of IFN-γ+ CD8+ T-cells produced by co-culture of BMDCs and T-cells in the absence of B16-Ova cells. Error bars indicate SEM. * indicates p<0.0001 when compared to cells treated with (−) using ANOVA followed by Dunnett's multiple comparisons test.

FIGS. 10A and 10B are diagrams depicting the therapeutic efficacy resulting from intra-tumoral administration of ex vivo chemotherapy-treated tumor cells in combination with systemic immune checkpoint blockade. Intra-tumoral injection of ex-vivo DNA damaging chemotherapy-treated tumor cells promotes effective DC-mediated T-cell priming and expansion when combined with systemic ICI (FIG. 10B), while intra-tumoral injection of free cytotoxic is ineffective (FIG. 10A). FIG. 10C is an illustration showing contact of tumor cells with cytotoxic drugs, e.g., etoposide/mitoxantrone, yields live, injured cells (AnnV−/DAPI−) and dead cells (AnnV+ and/or DAPI+).

### DETAILED DESCRIPTION OF THE INVENTION

### I. Definitions

As used herein, the term “cellular vaccine”, generally refers to a therapeutic agent against cancer and contains immunogenic isolated activated tumor cells.

As used herein the terms “treatment” or “treating” refer to administering a composition to a subject or a system to treat one or more symptoms of a disease. The effect of the administration of the composition to the subject can be, but is not limited to, the cessation of a particular symptom of a condition, a reduction or prevention of the symptoms of a condition, a reduction in the severity of the condition, the complete ablation of the condition, a stabilization or delay of the development or progression of a particular event or characteristic, or minimization of the chances that a particular event or characteristic will occur.

As used herein the terms “prevent”, “preventing”, “prevention” refers to reduction in recurrence of a particular symptom, adverse condition, disorder, or disease in a clinically asymptomatic individual who is at risk of developing, is susceptible to, or is predisposed to a particular adverse condition, disorder, or disease.

As used herein, the term “recurrence” refers to emergence of a tumor, usually after a period of time during which the cancer could not be detected. The cancer may come back to the same place as the original (primary) tumor or to another place in the body.

As used herein, the term “isolated”, in the context of cells, refers to cells extracted from a location in a patient. The isolated cells may be isolated by, for example, biopsy, aspiration, blood draw, and the like.

As used herein, the term “primary”, in the context of cells, refers to cells taken directly from living tissue (e.g. biopsy material) and established for growth ex vivo.

As used herein, the term “ex vivo,” refers to a manipulation done in or on tissue such as cells from an organism in an external environment. In ex vivo manipulations, an organism supplies the tissue whereas in in vitro manipulations, a cell line is used.

As used herein, the term “activated”, in the context of cells, refers to cancer cells treated with one or more genotoxic drug(s) and having an immunogenic state. Typically, activated cells include a degree of DNA damage holding the activated cells in a state of growth arrest, necrosis, necroptosis, and/or apoptosis. Activated cells may additionally or alternatively include an increase in RIPK1 and/or activated NF-κB signaling.

As used herein, the term “genotoxic drug” refers to a chemical agent that damages the genetic information within a cell. In the context of activating cells, genotoxic drugs include genotoxic chemotherapy agents used in treating cancer. Examples include alkylating agents that interfere with DNA replication and transcription by modifying DNA bases (such as busulfan, carmustine, mechlorethamine), intercalating agents that interfere with DNA replication and transcription by wedging themselves into the spaces in between DNA's nucleotides (such as daunorubicin, doxorubicin, epirubicin), and enzyme inhibitors that inhibit enzymes that are crucial to DNA replication (decitabine, etoposide, irinotecan).

As used herein, the term “necroptosis” refers to the art recognized programmed form of necrosis, or inflammatory cell death. During necroptosis, the cells undergo “cellular suicide” in a caspase-independent fashion. Unlike in apoptosis, necrosis and necroptosis do not involve caspase activation. Necrotic cell death culminates in leakage of cell contents into the extracellular space, in contrast to the organized disposal of cellular contents into apoptotic bodies.

As used herein, the term “autologous” refers to tissues, cells, or biological material taken from individual's own tissues or cells.

As used herein, the term “allogeneic” refers to tissues, cells, or biological material taken from different individuals of the same species.

As used herein, the term “immunogenic”, in the context of a cell state, refers to a cell state capable of increasing the percentage of CD3+CD8+IFNγ+ T cells in vitro, ex vivo, and/or in vivo. The cell state capable of increasing the percentage of CD3+CD8+IFNγ+ T cells in vitro, ex vivo, and/or in vivo generally includes changes in one or more cell death markers over the same markers in control cells. The changes in the one or more cell death markers include increase in calreticulin externalization, activation of RIPK1, secretion of High mobility group box 1 (HMGB1) and secretion of ATP when compared to the same markers in the control cells.

The term ‘T cell” refers to a CD4+ T cell or a CD8+ T cell. The term T cell includes TH1 cells, TH2 cells and TH17 cells.

The term “T cell cytotoxicity” includes any immune response that is mediated by CD8+ T cell activation. Exemplary immune responses include cytokine production, CD8+ T cell proliferation, granzyme or perforin production, clearance of an infectious agent, and/or a cancerous cell.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

The terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

The term “effective amount” or “therapeutically effective amount” means a dosage sufficient to provide treatment for a disorder, disease, or condition being treated, to induce or enhance an immune response, or to otherwise provide a desired pharmacologic and/or physiologic effect. The precise dosage will vary according to a variety of factors such as subject-dependent variables (e.g., age, immune system health, etc.), the disease, the disease stage, and the treatment being effected.

As used herein, the term “antibody” refers to both polyclonal and monoclonal antibodies. In addition to intact immunoglobulin molecules, also included are fragments or polymers of those immunoglobulin molecules, and human or humanized versions of immunoglobulin molecules or fragments thereof. The antibodies can be tested for their desired activity using the in vitro assays, or by analogous methods, after which their in vivo therapeutic and/or diagnostic activities can be confirmed and quantified according to known clinical testing methods.

As used herein, the terms “binding fragment,” “antigen binding fragment,” “antibody binding fragment,” and the like, refer to one or more portions of an antibody that contain the antibody's CDRs and, optionally, the framework residues that comprise the antibody's “variable region” antigen recognition site, and exhibit an ability to immunospecifically bind antigen. Such fragments include Fab′, F(ab′)2, Fv, single chain (ScFv), etc., and mutants and variants thereof, naturally occurring variants. As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

As used herein the terms “inhibit” and “reduce” refer to reducing or decreasing activity, expression, or a symptom. This can be a complete inhibition or reduction of in activity, expression, or a symptom, or a partial inhibition or reduction. Inhibition or reduction can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% reduction in activity, expression, or a symptom relative to a control.

As used herein, the phrase “not substantially” specifies a reduction or inhibition of no more than 25%, 20%, 15%, 12.5%, 10%, 5%, 4%, 3%, 2%, or 1%.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II. Cellular Vaccines

Described are cellular vaccines for treating cancer and/or preventing recurrence of cancer. Typically, the vaccines include isolated activated tumor cells. The cellular vaccines can activate cytotoxic immune response against the cancer cells in vivo, induce tumor regression, enhance survival from cancer, or a combination thereof. Additionally, or alternatively, the vaccines may prevent tumor recurrence, for example, for a period of about 5 years to about 10 years, such as for at least 5 years, for at least 6 years, for at least 7 years, for at least 8 years, for at least 9 years, or for at least 10 years. Additionally, or alternatively, the vaccines may induce a long-lasting anti-tumor immunological memory.

The vaccines may include immune checkpoint inhibitors (ICI), non-cellular cancer antigens, adjuvants, and pharmaceutically acceptable carriers.

In some aspects, the vaccines may include antigen presenting cells (APCs) and T cells, including antigen-primed cytotoxic T cells.

A. Cells

The cells in the cellular vaccine include isolated, activated tumor cells. In some aspects, the vaccines may also include APCs and/or T cells.

1. Isolated Cells

Typically, the cellular vaccine includes tumor cells isolated from a subject with cancer. The isolated tumor cells are typically activated tumor cells. Typically, the cells are primary cells taken directly from living tissue (e.g. biopsy material) and established for growth ex vivo. Preferably, the cells are not cells that have undergone an ex vivo immortalization process. Thus, in preferred embodiments, the isolated cells are not a cell line e.g., an immortalized cell line.

a. Transfected Cells

The isolated cell may, but need not necessarily, be transformed or transfected ex vivo. For example, in some embodiments, the isolated cells are transformed or transfected with a genetic expression construct while being cultured ex vivo. The genetic expression constructs may express a nucleic acid of interest, such as a nucleic acid encoding one or more cytokines, chemokines, signaling molecules, and transcription factors. For example, the genetic expression constructs may express cytokines, such as IL-2, chemokines, such as GM-CSF, signaling molecules that function downstream of RIPK1 kinase, or NF-κB transcription factors.

Genetic constructs typically include an expression control sequence operably linked to and a nucleic acid of interest. The genetic construct can be expressed extrachromosomally, or integrated in the cell's genome.

Nucleic acids encoding chemokines, cytokines, signaling molecules or transcription factors can be inserted into vectors for expression in cells. As used herein, a “vector” is a replicon, such as a plasmid, phage, virus or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors and integrated into the genome can be operably linked to one or more expression control sequences. For example, the control sequence can be incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

An expression vector can include a tag sequence. Tag sequences are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, Conn.), maltose E binding protein and protein A.

Vectors containing nucleic acids to be expressed can be transferred into activated tumor cells. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection.

The vectors can be used to express one or more cytokines, chemokines, signaling molecules, and transcription factors in cells. An exemplary vector includes, but is not limited to, an adenoviral vector. One approach includes nucleic acid transfer into primary cells in culture followed by autologous transplantation of the ex vivo transformed cells into the host, either systemically or into a particular organ or tissue. Ex vivo methods can include, for example, the steps of harvesting cells from a subject, culturing the cells, transducing them with an expression vector, and maintaining the cells under conditions suitable for expression of the encoded polypeptides. These methods are known in the art of molecular biology. The transduction step can be accomplished by any standard means used for ex vivo gene therapy, including, for example, calcium phosphate, lipofection, electroporation, viral infection, and biolistic gene transfer. Alternatively, liposomes or polymeric microparticles can be used. Cells that have been successfully transduced then can be selected, for example, for expression of the coding sequence or of a drug resistance gene. The cells then can be lethally irradiated (if desired) and injected or implanted into the subject. In one embodiment, expression vectors containing nucleic acids encoding fusion proteins are transfected into cells that are administered to a subject in need thereof.

Nucleic acids may also be administered in vivo by viral means. Nucleic acid molecules encoding polypeptides or fusion proteins may be packaged into retrovirus vectors using packaging cell lines that produce replication-defective retroviruses, as is well-known in the art. Other virus vectors may also be used, including recombinant adenoviruses and vaccinia virus, which can be rendered non-replicating. In addition to naked DNA or RNA, or viral vectors, engineered bacteria may be used as vectors.

Nucleic acids may also be delivered by other carriers, including liposomes, polymeric micro- and nanoparticles and polycations such as asialoglycoprotein/polylysine.

In addition to virus- and carrier-mediated gene transfer in vivo, physical means well-known in the art can be used for direct transfer of DNA, including administration of plasmid DNA and particle-bombardment mediated gene transfer.

b. Non-Transfected Cells

In some embodiments, the isolated cells are not genetically modified by transformation or transfection of a genetic construct expression of which induces cell death. In some embodiments, the isolated cells are not genetically modified by transformation or transection of any genetic construct. Thus, in some embodiments activated tumor cells do not include a heterologous genetic construct, e.g., an introduced nucleic acid construct for overexpression of an endogenous protein, or encoding a product not found in the cells, following isolation from the tumor.

c. Cell Dose and Cell Treatment

Typically, the vaccine contains between about 10⁴ and 10⁹ isolated and activated cells per injection dose. Generally, the vaccine may contain any number of isolated activated cells in this range, such as about 10⁴, about 10⁵, about 10⁶, about 10⁷, about 10⁸, or about 10⁹ cells. Preferred ranges include between about 10⁴ and 10⁷, such as between about 10⁴ and 1×10⁶, between about 10⁴ and 2×10⁶, between about 10⁴ and 3×10⁶, between about 10⁴ and 4×10⁶, between about 10⁴ and 5×10⁶, between about 10⁴ and 6×10⁶, between about 10⁴ and 7×10⁶, between about 10⁴ and 8×10⁶, between about 10⁴ and 9×10⁶, between about 10⁴ and 10×10⁶ isolated activated cells per injection dose.

Tumor cells may be isolated from a tumor of subject suffering from breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, head and neck cancer, cervical cancer, maxillary sinus cancer, bladder cancer, esophageal cancer, Hodgkin's disease, or adrenocortical cancer.

The isolated cells are typically treated with genotoxic drugs to produce activated cells. Typically, a sample of isolated cells is cultured in the presence of a genotoxic drug for a period of time. The period of time may be between about 1 hour and 48 hours (h), such as about 3 h, 6 h, 9 h, 12 h, 15 h, 18 h, 21 h, 24 h, 27 h, 30 h, 33 h, 36 h, 29 h, 42 h, 45 h, or 48 h.

The genotoxic drug is typically an anti-neoplastic agent, such as a chemotherapy drug. Suitable genotoxic drugs include, but are not limited to, alkylating agents (such as cisplatin, carboplatin, oxaliplatin, mechlorethamine, cyclophosphamide, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and docetaxel, epothilones A-F, and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), and combinations thereof.

Other suitable anti-neoplastic agents that may be used to activate cells include actinomycin, carmustine (BCNU), methyl-CCNU, camptothecin and derivatives thereof, phenesterine, paclitaxel and derivatives thereof, docetaxel and derivatives thereof, tamoxifen, piposulfan, altretamine, asparaginase, busulfan, carboplatin, carmustine, cladribine, cyclophosphamide, cytarabine, dacarbazine, diethylstilbestrol, ethinyl estradiol, mitotane, mitoxantrone, paclitaxel, pentastatin, pipobroman, prednisone, procarbazine, streptozocin, and tamoxifen.

In particular embodiments, the genotoxic drug is etoposide or mitoxantrone or doxorubicin.

The experiments below also show that mitogen-activated protein kinase 2 (MK2) inhibitor enhances BMDC-mediated T-cell priming. Thus, in some embodiments, cells are treated with MK2 inhibitor. The cells are most typically treated with the MK2 inhibitor ex vivo as part of the activation step(s).

The isolated cells treated with the genotoxic drug and optional MK2 inhibitor are induced to form activated, immunogenic cells. The activated cells typically have genomic DNA damage, and may initiate one or more programmed cell-death pathways. Thus, the activated tumor cells can be non-proliferative.

The experiments below show that chemotherapy-induced cell stress signaling in live injured cells, but not the presence of dead cells, was the primary determinant of T-cell immunity. This effect seems to be mediated by RIPK1, p38MAPK and NF-kB signaling in the injured tumor cells. Furthermore, results show that direct intra-tumoral injection of ex vivo chemotherapy treated cells as an injured cell adjuvant, in combination with systemic ICI drives anti-tumor immunity and tumor regression.

The activated cells may have markers of apoptosis or necroptosis. Typically, the immunogenic cells are cells with cellular markers of necroptosis. These include DNA damage, calreticulin externalization, and activation of Receptor-Interacting Protein Kinase 1 (RIPK1). The cells preferably have activated NF-κB signaling. In some embodiments, the NF-κB signaling is not substantially reduced compared to unactivated cells. In some embodiments, NF-κB signaling is increased compared to unactivated cells.

Nonetheless, the cells are typically injured live cells, rather than dead cells. In some embodiments, live cells are annexin V (“AnnV”) and DAPI double negative and dead cells are AnnV and/or DAPI single or double positive.

d. DNA Damage

Generally, the activated cells have DNA damage resulting in cessation of replication. The DNA damage typically includes DNA base modifications, intercalated agents wedged into the spaces in between DNA's nucleotides, single strand breaks, double strand breaks, and interstrand cross-links, blocking DNA replication.

DNA damage may also result from the cells activating programmed cell-death pathways apoptosis or necroptosis. The DNA damage may be detected by assessing the treated cells for DNA damage. The assessment may be done by any suitable method used in the art to assess DNA damage. Exemplary methods include cellular assays (such as flow cytometry, staining, or immunostaining using DNA-binding dyes (such as DAPI (4′,6-diamidino-2-phenylindole), Hoechst 33342, or antibodies binding damaged DNA, or commercially available kits for detecting DNA damage with staining or Enzyme-Linked Immunosorbent Assay (ELISA)), nucleic acid electrophoresis, hybridization assays, polymerase chain reaction (PCR), and spectrophotometry.

e. DNA Damage Repair Signaling

In the experiments below, live cell fractions from specific chemotherapy-treated B16-Ova cell mixtures showed phosphorylation of substrates of ATM and ATR and also for phospho-p38MAPK, and inhibition of specific DNA-damage signaling pathways in etoposide-treated B16-Ova cells impairs dendritic-cell mediated T-cell activation. The protein kinase ataxia-telangiectasia mutated (ATM) is best known for its role as an apical activator of the DNA damage response in the face of DNA double-strand breaks (DSBs). Following induction of DSBs, ATM mobilizes one of the most extensive signaling networks that responds to specific stimuli and modifies directly or indirectly a broad range of targets. Serine/threonine-protein kinase ATR also known as ataxia telangiectasia and Rad3-related protein (ATR) or FRAP-related protein 1 (FRP1) is a serine/threonine-specific protein kinase that is involved in sensing DNA damage and activating the DNA damage checkpoint, leading to cell cycle arrest. ATR is activated in response to persistent single-stranded DNA, which is a common intermediate formed during DNA damage detection and repair.

These results indicate that having intact and/or active DNA damage signaling may be important in activated cells. Thus, in some embodiments, the activated cells include one or more active DNA damage signaling pathways, which may be induced, activated, or increased by single or double DNA strand breaks, are induced by the genotoxic agent. In some embodiments, signaling is mediated and/or evidenced by phosphorylation of p38MAPK, an ATM and/or ATR substrate (e.g., phospho-S), or a combination thereof. Activated cells may have an increase in phosphorylated p38MAPK, an ATM and/or ATR substrate (e.g., phospho-S), or a combination thereof following treatment with the genotoxic agent.

f. Calreticulin Externalization

The activated cells may have a translocation of calreticulin from intracellular stores onto the cell surface, an event referred to as calreticulin externalization.

Calreticulin is a highly conserved chaperone protein of the endoplasmatic reticulum (ER) that has specificity towards glycoprotein substrates. Calreticulin is important for the assembly and cell surface expression of MHC class I molecules and hence for CD8 T cell recognition of antigens presented by MHC class I molecules. Calreticulin is a structural homolog of the ER chaperone calnexin, although calnexin is membrane-anchored, whereas calreticulin is soluble. Calreticulin contains a highly acidic C-terminal region (residues 351-359) that binds multiple calcium ions with low affinity. The counterpart of this region is absent in the lumenal domains of calnexin. The acidic C-terminus of calreticulin is important for maintenance of cellular calcium homeostasis, and cells deficient in calreticulin have reduced calcium storage capacity in the ER. In mice, total calreticulin deficiency is embryonic lethal due to alterations in cellular calcium homeostasis. The acidic region of calreticulin also plays a role in ER-retention of the protein. Calreticulin translocates to the cell surface under conditions of cell stress and tumorigenesis, and cell-surface calreticulin is an “eat-me” signal (Raghavan et al., Trends Immunol, 34(1):13-21 (2013)).

Thus, in some embodiments, the isolated activated cells include cells having externalized calreticulin (CALR+). Typically, the isolated activated cells have a greater percentage of CALR+cells than isolated cells treated under control conditions (such as cells cultured under the same conditions and for the same length of time as the isolated and activated cells, but without the genotoxic agent). The increase in the number of CALR+cells may be an increase by at least about 2 fold, 3 fold, 4 fold, 5 fold, 6 fold, 7 fold, 8 fold, 9 fold, 10 fold, 11 fold, 12 fold, 13 fold, 14 fold, 15 fold, 20 fold, 30 fold, 40 fold, 50 fold, or more fold. For example, the isolated cells treated under control condition may have about 1% CALR+cells when measured by flow cytometry, while the isolated cells treated with a genotoxic drug may have at least about 2%, 3%, 4%, 5%, 6%, 7%, 8%, 9%, 10%, 11%, 12%, 13%, 14%, 15%, 20%, 30%, 40%, 50% or more percent CALR+cells, showing a respective fold increase. Typically, the population of activated cells has between about 1% and about 100% of the cells with externalized calreticulin, such as between about 5% and about 100%, between about 5% and about 90%, between about 7.5% and about 100%, between about 10% and about 100%, between about 15% and about 100%, between about 17.5% and about 100%, between about 20% and about 100%, between about 25% and about 100%, between about 30% and about 100%, between about 40% and about 100%, or between about 50% and about 100% of the cells with externalized calreticulin. The increase may be detected using flow cytometry or immunostaining assays.

g. RIPK1 and/or NF-κB Signaling

Typically, at least a portion of the isolated activated cells have activated (phosphorylated) receptor interacting protein kinase 1, activated nuclear factor kappa-light-chain-enhancer of activated B cells (NF-κB), or a combination thereof.

In necroptosis, receptor interacting protein kinase 1 (RIP, RIP1 or RIPK1) and RIPK3 serve as key signaling effectors. These two protein serine/threonine kinases interact with one another via their RIP homotypic interaction motif. This results in phosphorylation of both RIPK1 and RIPK3, leading to recruitment and activation of the mixed lineage kinase domain like (MLKL) protein. Once activated, MLKL translocates to and disrupts the plasma membrane. Loss of membrane integrity during necroptosis results in the release of cellular contents, leading to inflammatory responses (Zhang et al., Cell Death Dis., 10(3):245 (2019)).

The results below also show that inhibition of NF-κB signaling can reduce genotoxic drug-induced tumor cell immunogenicity. Thus, preferably, NF-κB signaling is active or activated. In some embodiments NF-κB signaling is not substantially reduced in the activated tumor cells compared to unactivated cells. In some embodiments NF-κB signaling is increased in the activated tumor cells compared to unactivated cells. The unactivated cells may be, for example, the same tumor cells without genotoxic drug treatment, or treated with a different, non-activating drug or drug dose.

Activation of the NF-κB is typically initiated by the signal-induced degradation of inhibitory IκB proteins. This occurs primarily via activation of IκB kinase (IKK). IKK is composed of a heterodimer of the catalytic IKKα and IKKβ subunits NEMO (NF-κB essential modulator) or IKKγ. When activated by signals the IκB kinase phosphorylates two serine residues located in an IκB regulatory domain. When phosphorylated on these serines (e.g., serines 32 and 36 in human IκBα), the IκB proteins are modified by ubiquitination, which then leads them to be degraded by a cell structure called the proteasome. With the degradation of IκB, cytosolic NF-κB complex is then freed to enter the nucleus where it can induce target gene expression.

The RIPK1 and/or NF-κB activation in the isolated activated cells may be detected using protein interrogation methods (such as Western blotting, immunoprecipitation, and pull down assays) and cell staining methods, such as immunostaining. Translocation of NF-κB to nucleus can be detected immunocytochemically and/or measured by flow cytometry.

Typically, the isolated activated cells have a greater percentage of cells with activated RIPK1 than isolated cells treated under control conditions (such as cells cultured under the same conditions and for the same length of time as the isolated and treated cells, but without the genotoxic agent).

In some embodiments, the isolated activated cells additionally or alternatively have the same or a greater percentage of cells with activated NF-κB as isolated cells treated under control conditions (such as cells cultured under the same conditions and for the same length of time as the isolated and treated cells, but without the genotoxic agent), and/or a greater percentage of cells with activated NF-κB than isolated cells treated under NF-κB inhibitory conditions (such as cells cultured under the same conditions and for the same length of time as the isolated and genotoxic agent-only treated cells, but with the genotoxic agent in combination with an NF-κB inhibitor).

The increase in the number of cells with activated RIPK1, activated NF-κB, or a combination thereof may be an increase by at least about 2 fold, 3 fold, 4 fold, 5 fold, 6 fold, 7 fold, 8 fold, 9 fold, 10 fold, 11 fold, 12 fold, 13 fold, 14 fold, 15 fold, or more fold when measured by Western blotting.

2. Antigen Presenting Cells

The vaccines may include professional antigen presenting cells (APCs). The APCs may be autologous or allogeneic.

APCs include cells that displays antigen complexed with major histocompatibility complexes (MHCs) on their surfaces; this process is known as antigen presentation. T cells may recognize these complexes using their T cell receptors (TCRs). APCs process antigens and present them to T cells. Professional APCs express MHC class I and MHC class II molecules and can stimulate CD4+ helper T cells as well as CD8+ cytotoxic T cells. Professional antigen-presenting cells include macrophages, B cells, and dendritic cells. Preferred APCs in the vaccine include macrophages and dendritic cells. Most preferred APCs in the vaccine include autologous dendritic cells.

The APCs may be at the same cell number as the isolated activated cells in the vaccine, at a greater number than the isolated activated cells in the vaccine, or at a lower number than the isolated activated cells in the vaccine.

For example, the vaccine may contain any number of APCs in the range between about 10⁴ and 10⁸ cells per injection dose, such as about 10⁴, about 10⁵, about 10⁶, about 10⁷, or about 10⁸ APCs. Preferred ranges include between about 10⁴ and 10⁷, such as between about 10⁴ and 1×10⁶, between about 10⁴ and 2×10⁶, between about 10⁴ and 3×10⁶, between about 10⁴ and 4×10⁶, between about 10⁴ and 5×10⁶, between about 10⁴ and 6×10⁶, between about 10⁴ and 7×10⁶, between about 10⁴ and 8×10⁶, between about 10⁴ and 9×10⁶, between about 10⁴ and 10×10⁶ of APCs per injection dose.

3. T Cells

The vaccines may include cytotoxic T cells. The cytotoxic T cells may be autologous or allogeneic.

The cytotoxic T cells (also referred to as CD8+ T-cell or killer T cell) are T lymphocytes that kill cancer cells, cells that are infected (particularly with viruses), or cells that are damaged in other ways. Most cytotoxic T cells express T-cell receptors (TCRs) that can recognize a specific antigen. An antigen is a molecule capable of stimulating an immune response, and is often produced by cancer cells or viruses. Antigens inside a cell are bound to class I MHC molecules, and brought to the surface of the cell by the class I MHC molecule, where they can be recognized by the T cell. If the TCR is specific for that antigen, it binds to the complex of the class I MHC molecule and the antigen, and the T cell destroys the cell.

In order for the TCR to bind to the class I MHC molecule, the former must be accompanied by a glycoprotein called CD8, which binds to the constant portion of the class I MHC molecule. Therefore, these T cells are called CD8+ T cells.

The affinity between CD8 and the MHC molecule keeps the cytotoxic T cell and the target cell bound closely together during antigen-specific activation. CD8+ T cells are recognized as cytotoxic T cells once they become activated and are generally classified as having a pre-defined cytotoxic role within the immune system. However, CD8+ T cells also have the ability to make some cytokines. Once activated, the TC cell undergoes clonal expansion with the help of the cytokine Interleukin-2 (IL-2), which is a growth and differentiation factor for T cells. This increases the number of cells specific for the target antigen that can then travel throughout the body in search of antigen-positive somatic cells.

The T cells in the vaccine may be naive CD8+ T cells or primed CD8+ T cells. The first contact of a T cell with its specific antigen is generally known as priming and causes differentiation into effector T cells. Priming of naïve T cells requires dendritic cell antigen presentation. Priming of naive CD8 T cells generates cytotoxic T cells capable of directly killing antigen-containing cells.

The T cells may be present in the vaccine at the same number as, or less than, the number of APCs per injection dose. The T cells may be present at number between about 10⁴ and 10⁸ cells per injection dose, such as about 10⁴, about 10⁵, about 10⁶, about 10⁷, or about 10⁸ cells. Preferred ranges include between about 10⁴ and 10⁷, such as between about 10⁴ and 1×10⁶, between about 10⁴ and 2×10⁶, between about 10⁴ and 3×10⁶, between about 10⁴ and 4×10⁶, between about 10⁴ and 5×10⁶, between about 10⁴ and 6×10⁶, between about 10⁴ and 7×10⁶, between about 10⁴ and 8×10⁶, between about 10⁴ and 9×10⁶, between about 10⁴ and 10×10⁶ of T cells per injection dose.

B. Immune Checkpoint Inhibitors

The cellular vaccines may include one ore more immune checkpoint inhibitors (ICI). Generally, the ICI include small molecules, antibodies, or an antibody fragment against programmed cell death protein 1 (PD-1), against PD-1 Ligand 1 (PD-L1), and against cytotoxic T-lymphocyte-associated antigen 4 (CTLA-4).

Typically, the vaccines include ICI at between about between about 0.1 mg/kg and about 100 mg/kg of the body weight of the patient in an injection dose. Suitable amounts of the ICI in the vaccine include between about 0.1 mg/kg and about 500 mg/kg, between about 0.1 mg/kg and about 250 mg/kg, between about 0.1 mg/kg and about 100 mg/kg, between about 0.1 mg/kg and about 80 mg/kg, and between about 0.1 mg/kg and about 60 mg/kg, such as between about 0.5 mg/kg and about 20 mg/kg, or between about 1 mg/kg and about 10 mg/kg. Specific concentrations of the ICI include 0.1 mg/kg, 0.2 mg/kg, 0.3 mg/kg 0.4 μM, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, and 50 mg/kg.

1. PD-1 Antagonists

Activation of T cells normally depends on an antigen-specific signal following contact of the T cell receptor (TCR) with an antigenic peptide presented via the major histocompatibility complex (MHC) while the extent of this reaction is controlled by positive and negative antigen-independent signals emanating from a variety of co-stimulatory molecules. The latter are commonly members of the CD28/B7 family Conversely, Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some embodiments, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example, Freeman, Proc. Nall. Acad. Sci. U. S. A, 105: 10275-10276 (2008)).

Therefore, proteins, antibodies or small molecules that prevent co-ligation of PD-1 and TCR on the T cell membrane are also useful PD-1 antagonists.

Other PD-1 antagonists include antibodies that bind to PD-1 or ligands of PD-1, and other antibodies.

Suitable anti-PD-1 antibodies include, but are not limited to, those described in the following publications: PCT/IL03/00425 (Hardy et al, WO/2003/099196), PCT/JP2006/309606 (Korman et al, WO/2006/121168), PCT/US2008/008925 (Li et al, WO/2009/014708), PCT/JP03/08420 (Honjo et al, WO/2004/004771), PCT/JP04/00549 (Honjo et al, WO/2004/072286), PCT/IB2003/006304 (Collins et al, WO/2004/056875), PCT/US2007/088851 (Ahmed et al, WO/2008/083174), PCT/US2006/026046 (Korman et al, WO/2007/005874), PCT/US2008/084923 (Terrett et al, WO/2009/073533), and Berger et al, Clin. Cancer Res., 14(10):3044-51 (2008).

A specific example of an anti-PD-1 antibody is MDX-1106 (see Kosak, US 20070166281 (pub. 19 Jul. 2007) at par. 42), a human anti-PD-1 antibody, preferably administered at a dose of 3 mg/kg.

Exemplary anti-B7-H1 antibodies include, but are not limited to, those described in the following publications: PCT/US06/022423 (WO/2006/133396, pub. 14 Dec. 2006), PCT/US07/088851 (WO/2008/083174, pub. 10 Jul. 2008) US 2006/0110383 (pub. 25 May 2006)

A specific example of an anti-B7-H1 antibody is MDX-1105 (WO/2007/005874, published 11 Jan. 2007)), a human anti-B7-Hl antibody.

For anti-B7-DC antibodies see U.S. Pat. Nos. 7,411,051, 7,052,694, 7,390,888, and U.S. Published Application No. 2006/0099203.

The antibody can be a bi-specific antibody that includes an antibody that binds to the PD-1 receptor bridged to an antibody that binds to a ligand of PD-1, such as B7-H1. In some embodiments, the PD-1 binding portion reduces or inhibits signal transduction through the PD-1 receptor.

Other exemplary PD-1 receptor antagonists include, but are not limited to B7-DC polypeptides, including homologs and variants of these, as well as active fragments of any of the foregoing, and fusion proteins that incorporate any of these. In a preferred embodiment, the fusion protein comprises the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, preferably from mouse or primate, preferably human, wherein the fragment binds to and blocks PD-1 but does not result in inhibitory signal transduction through PD-1. The fragments can also be part of a fusion protein, for example an Ig fusion protein.

Other useful polypeptides PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al, Immunity, Vol. 27, pp. 1 11-122, (2007)). Such fragments also include the soluble ECD portion of the PD-1 protein that includes mutations, such as the A99L mutation, that increases binding to the natural ligands (Molnar et al, PNAS, 105: 10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al, J. Clin. Invest. 119(8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

2. CTLA-4 Antagonists

Other molecules useful in mediating the effects of T cells in an immune response are also contemplated as active agents. For example, in some embodiments, the molecule is an agent binds to CTLA4.

Dosages for anti-PD-1, anti-B7-Hl, and anti-CTLA4 antibody, are known in the art and can be in the range of 0.1 to 100 mg/kg, with shorter ranges of 1 to 50 mg/kg preferred and ranges of 10 to 20 mg/kg being more preferred. An appropriate dose for a human subject is between 5 and 15 mg/kg, with 10 mg/kg of antibody (for example, human anti-PD-1 antibody, like MDX-1106).

Specific examples of an anti-CTLA4 antibody useful in the methods of the invention are Ipilimumab, also known as MDX-010 or MDX-101, a human anti-CTLA4 antibody, preferably administered at a dose of about 10 mg/kg, and Tremelimumab a human anti-CTLA4 antibody, preferably administered at a dose of about 15 mg/kg. See also Sammartino, et al, Clinical Kidney Journal, 3(2): 135-137 (2010), published online December 2009.

In other embodiments, the antagonist is a small molecule. A series of small organic compounds have been shown to bind to the B7-1 ligand to prevent binding to CTLA4 (see Erbe et al, J. Biol. Chem., 277:7363-7368 (2002)). Such small organics could be administered alone or together with an anti-CTLA4 antibody to reduce inhibitory signal transduction of T cells.

C. Additional Cancer Antigens

The cellular vaccines may include additional cancer antigens that are not derived from the isolated activated cells. The additional cancer antigens may be nucleic acids, peptides, or proteins. The additional cancer antigens may be synthetic antigens or enriched or purified from cancer cells.

A cancer antigen is an antigen that is typically expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen can be MART-1/Melan-A, gp100, adenosine deaminase-binding protein (ADAbp), FAP, cyclophilin b, colorectal associated antigen (CRC)—0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-1, GAGE-2, GAGE-3, GAGE-4, GAGE-5, GAGE-6, GAGE-7, GAGE-8, GAGE-9, BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pmel117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, imp-1, PIA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, or c-erbB-2.

D. Adjuvants and Pharmaceutically Acceptable Carriers

The cellular vaccines may include one or more adjuvants and/or one or more pharmaceutically acceptable carriers.

1. Adjuvants

The adjuvant may be without limitation alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

Adjuvants may be TLR ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor.

2. Pharmaceutically Acceptable Carriers

Pharmaceutically acceptable carriers include compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. Pharmaceutically acceptable carriers include, but are not limited to, buffers, diluents, preservatives, binders, stabilizers, a mixture or polymer of sugars (lactose, sucrose, dextrose, etc.), salts, and combinations thereof.

The compositions may be administered in combination with one or more physiologically or pharmaceutically acceptable carriers, thickening agents, co-solvents, adhesives, antioxidants, buffers, viscosity and absorption enhancing agents and agents capable of adjusting osmolarity of the formulation. Proper formulation is dependent upon the route of administration chosen. If desired, the compositions may also contain minor amounts of nontoxic auxiliary substances such as wetting or emulsifying agents, dyes, pH buffering agents, or preservatives.

In a preferred embodiment, cell compositions are administered in an aqueous solution, by parenteral injection or infusion. The formulation may also be in the form of a suspension or emulsion. In general, pharmaceutical compositions are provided including effective amounts of the composition, and optionally include pharmaceutically acceptable diluents, preservatives, solubilizers, emulsifiers, adjuvants and/or carriers. Such compositions include diluents such as sterile water, buffered saline of various buffer content (e.g., Tris-HCl, acetate, phosphate), pH and ionic strength; and optionally, additives such as anti-oxidants (e.g., ascorbic acid, sodium metabisulfite), and preservatives and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate.

In some embodiments, the pharmaceutical composition for cells is a saline solution, preferably a buffered saline solution phosphate buffered saline or sterile saline, or tissue culture medium.

### III. Methods of Making the Cellular Vaccines

The vaccine is produced by isolating tumor cells from a patient and processing the tumor cells into a vaccine formulation ex vivo. The processing includes ex vivo culture of the tumor cells with genotoxic drug(s) to form activated cells. The activated cells are immunogenic cells. For example, they typically increase the frequency of tumor-specific cytotoxic T cells ex vivo and/or in vivo. The increase in frequency of tumor-specific cytotoxic T cells may be measured ex vivo when co-cultured with dendritic cells and T cells, or in vivo, when injected into the patient's tumor, relative to the frequency of the tumor-specific cytotoxic T cells when control cells are co-cultured with dendritic cells and T cells under similar or the same conditions.

A. Isolating Tumor Cells

Typically, the subject's tumor cells are isolated from a tumor (for solid tumors) or from an aspirate or blood draw (for leukemia).

The tumor cells are typically isolated using biopsy, aspiration, blood draw, or other suitable techniques. The isolated cells may be cultured ex vivo to expand the number of cells. The isolated cells are typically treated to produce isolated activated cells. The activated cells may be highly immunogenic. The activated cells may be tested for immunogenicity markers to detect an increase in immunogenicity markers, such as calreticulin externalization, HMBG1 secretion, extracellular ATP, and/or activation of RIPK1 and/or NF-κB.

B. Incubation with Genotoxic Drug(s)

The isolated cells are typically treated with genotoxic drug(s) by incubating the cells under standard tissue culture conditions with genotoxic drug(s). Generally, the incubation includes culturing the isolated cells and the genotoxic drug(s) under standard tissue culture conditions for a period of time. Typically, the period of time for culture with the genotoxic drug(s) is between about 1 hour and 48 hours (h), such as about 3 h, 6 h, 9 h, 12 h, 15 h, 18 h, 21 h, 24 h, 27 h, 30 h, 33 h, 36 h, 29 h, 42 h, 45 h, or 48 h.

The genotoxic drug(s) may be used a concentration between about 0.1 μM and about 1000 μM. Suitable ranges for the concentration of the genotoxic drug(s) include between about 0.1 μM and about 500 μM, between about 0.1 μM and about 250 μM, between about 0.1 μM and about 100 μM, between about 0.1 μM and about 80 μM, and between about 0.1 μM and about 60 μM. Specific concentrations of the genotoxic drug(s) include 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, and 50 μM.

The experiments below show that the specific doses of mitoxantrone, etoposide, and doxorubicin that were maximally effective were not the doses that caused the greatest amount of cell death. Thus, the dosage of genotoxic drug used to generate activated cells is typically sufficient to injure the cells and induce stress signaling, but not sufficient to induce maximal cell death on a population of treated cells. Stress signaling can include DNA damage and repair pathways, including those involving ATM and ATR. For example, the experiments below show that 10 μM and 50 μM concentration of doxorubicin induced high levels of cell death, but were not effective at activating cells, while 0.5 μM and 1.0 μM concentration were effective at activating cells.

After treatment, the cells are typically washed (in some embodiments repeatedly) to remove the genotoxic drug(s).

The cells may then be assayed for immunogenicity.

Additionally or alternatively, the cells may be processed for packaging into injectable doses to form vaccines. Packaging may include preparing ampules, pre-loaded syringes, or capsules containing a dose of the vaccine for a single injection (injection dose).

The populations of cells used in the disclosed compositions and methods typically include injured, live cells, and are not typically composed entirely of dead cells. In some embodiments, an integer percent between 1-100 inclusive, of total cells are live, injured cells. For example, in some embodiments, 10%, 20%, 30%, 40%, 50%, 60%, 70%, 75%, 80%, 85%, 90%, 95%, or 100% of the cells are injured, live cells. In some embodiments, live, injured cells are cells that remain adherent to a substrate following treatment with the genotoxic agent. Floating and/or suspended cells may be discarded as dead cells. In some embodiments, live cells are annexin V (“AnnV”) and DAPI double negative and dead cells are AnnV and/or DAPI single or double positive.

The cells may be assayed for intact, induced, and/or increased DNA damage signaling. For example, in some embodiments, the cells show intact, induced, or increased activation of ATM and/or ATR substrates and/or phosphorylated p38MAPK after treatment with the genotoxic agent, and/or have reduced activation or are inactive in the presence of ATM and/or ATR and/or DNA-dependent Protein Kinase (DNA-PK) inhibitors.

C. Incubation with MK2 Inhibitor(s)

The experiments below also show that mitogen-activated protein kinase 2 (MK2) inhibitor enhances BMDC-mediated T-cell priming. Thus, in some embodiments, cells are treated with one or more MK2 inhibitors. The cells are most typically treated with the MK2 inhibitor ex vivo as part of the activation step(s). The cells can be treated with the MK2 inhibitor at the same or different (e.g., before or after) times as the genotoxic drug. The treatment period may be, for example, 1 hour and 48 hours (h), such as about 3 h, 6 h, 9 h, 12 h, 15 h, 18 h, 21 h, 24 h, 27 h, 30 h, 33 h, 36 h, 29 h, 42 h, 45 h, or 48 h.

The amount of MK2 inhibitor is typically an amount that is effective to reduce expression and/or activity of MK2 in the cells. In some embodiments, for small molecule drugs, the MK2 inhibitor may be used in a concentration between about 0.1 μM and about 1000 μM. Suitable ranges for the concentration of the MK2 inhibitor drug(s) include between about 0.1 μM and about 500 μM, between about 0.1 μM and about 250 μM, between about 0.1 μM and about 100 μM, between about 0.1 μM and about 80 μM, and between about 0.1 μM and about 60 μM. Specific concentrations of the genotoxic drug(s) include 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, and 50 μM.

Suitable MK2 inhibitors include, but are not limited to, MK2-IN-1 hydrochloride (CAS No. 1314118-94-9), MK-2 Inhibitor III (CAS No. 1186648-22-5), MK2-IN-1 (CAS No. 1314118-92-7), CMPD1 (CAS No. 41179-33-3), PHA 767491 hydrochloride (CAS Number 942425-68-5), and PF 3644022 (CAS Number 1276121-88-0), as well as inhibitory RNA molecules complementary to any region of MK2 mRNA, and its transcription variants (such as Accession: NM_004759.5; Accession: NM_032960.4; Accession: NM_001204269.2; Accession: XM_017002810.1; Accession: XM_017001213.1; and Accession: XM_011541400.2) and their homologs having between 50 and 99% sequence homology with the mRNA, and its transcription variants.

After treatment, the cells may be washed (in some embodiments repeatedly) to remove the MK2 inhibitor. The cells may then be assayed for immunogenicity. Additionally or alternatively, the cells may be processed for packaging into injectable doses to form vaccines. Packaging may include preparing ampules, pre-loaded syringes, or capsules containing a dose of the vaccine for a single injection (injection dose).

D. Inclusion of ICI

The vaccines may also include ICI admixed with the treated cells or included in the packaging. The ICI may be present in the single injectable dose at a concentration between about 0.1 mg/kg and about 100 mg/kg of the body weight of the patient.

E. Screening to Identify Drug(s) inducing Immunogenic Activated Tumor Cells

Assays for screening genotoxic drug(s) for inducing immunogenic isolated activated cells are also provided. The assays typically include the steps of:

a) isolating tumor cells from a subject and culturing them in one or more separate vessels as separate samples of the isolated tumor cells;

b) treating each of the separate samples of the isolated tumor cells with one or more genotoxic drugs at one or more different concentrations/dosages for a period of time of at least 3 hours, but typically between about 3 hours and 48 hours, such as 24 hours;

c) optionally repeating step b) for as many genotoxic drugs as is desired or needed to be screened;

d) collecting the treated cells from each separate sample and washing to remove the drug, optionally removing some or all dead cells (e.g., floating or suspended cells, and/or cells AnnV and/or DAPI single or double positive; and

e) optionally analyzing the treated cells for presence of immunogenic cell death markers,

to identify the drug that produced immunogenic activated cells.

Typically, analyzing includes subjecting the cells to any one of the flow cytometry, ELISA, cell viability, DNA damage testing, Western blotting and other suitable analyses generally known to those of skill in the art Immunogenic cell death markers include the levels of externalized calreticulin, increase in calreticulin externalization, activation of RIPK1, increase in secretion of HMGB1 and increase in secretion of ATP when compared to the same markers in control cells not treated with the drug. For example, activation of RIPK1 may be measured by Western blotting, while the levels of externalized calreticulin may be measuring using flow cytometry.

The drug that produces activated cells with the highest increase in immunogenic cell death markers may then identified as the drug that produces activated tumor cells with the highest immunogenic potential. Steps a)-e) may be repeated for different concentrations of genotoxic drug(s) to identify not just the drug, but also the best concentration at which the drug produces activated tumor cells with the highest immunogenic potential. The experiments below show that the specific doses of mitoxantrone, etoposide, and doxorubicin that were maximally effective were not the doses that caused the greatest amount of cell death. Thus, the dosage of genotoxic drug used to generate activated cells is typically sufficient to injure the cells and induce stress signaling, but not sufficient to induce maximal cell death on a population of treated cells.

Any of the assays discussed herein, including, but not limited to those exemplified in the experiments below, can be used in the identification and selection of drugs and dosages suitable for preparing cellular vaccines formed of isolated, activated, immunogenic tumor cells.

For example, the analysis to identify a drug or drugs that produced immunogenic activated cells may also include cross-presentation assays.

F. Cross-presentation Assay with Antigen Presenting Cells and T Cells

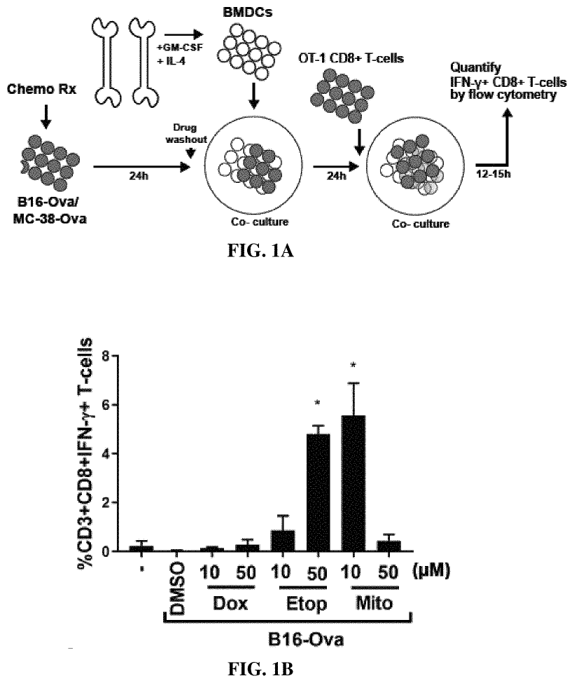

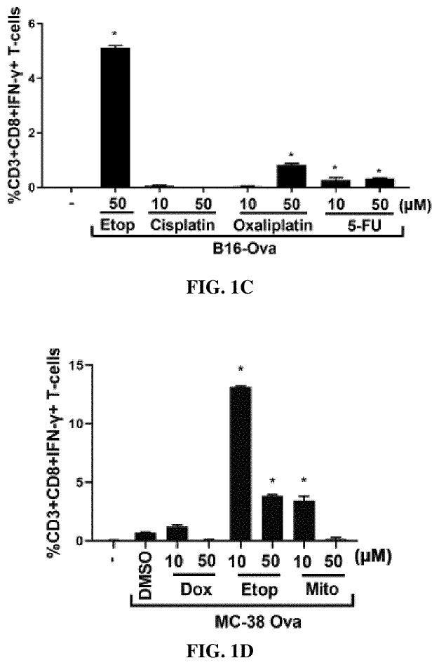

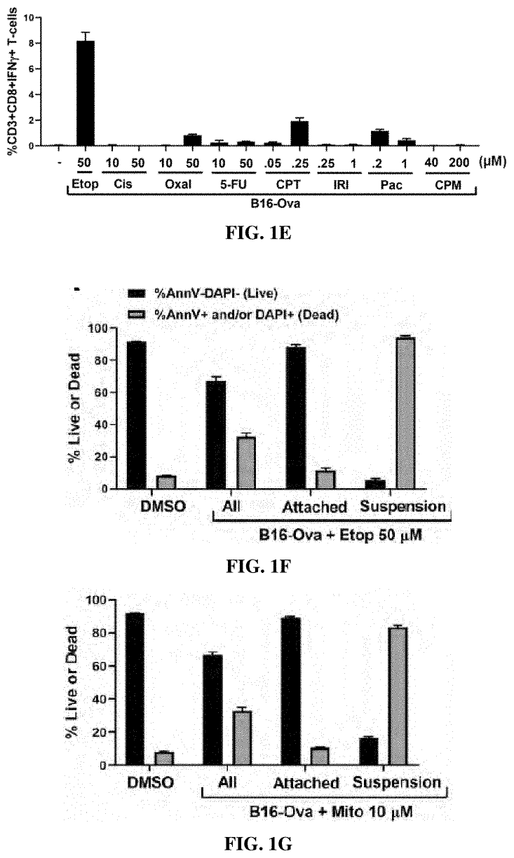

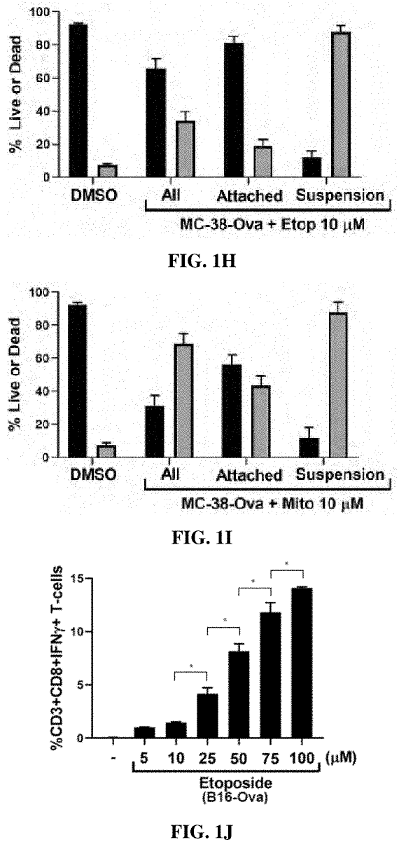

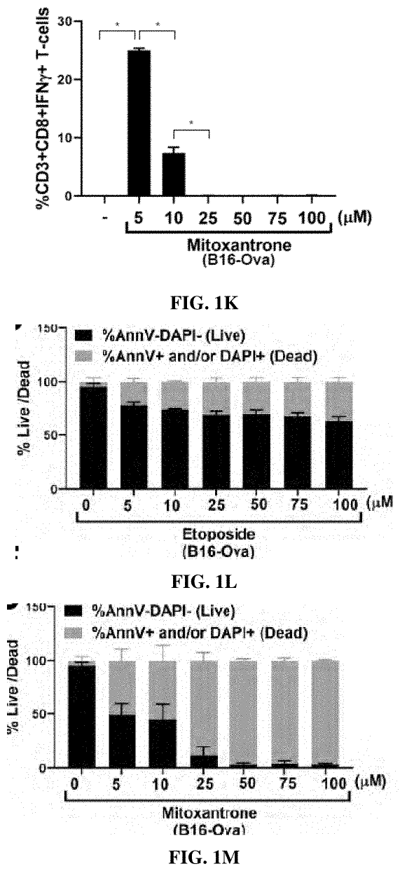

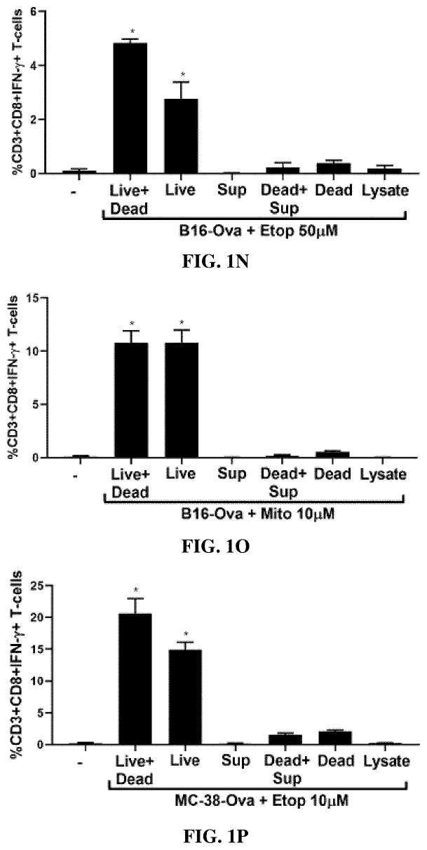

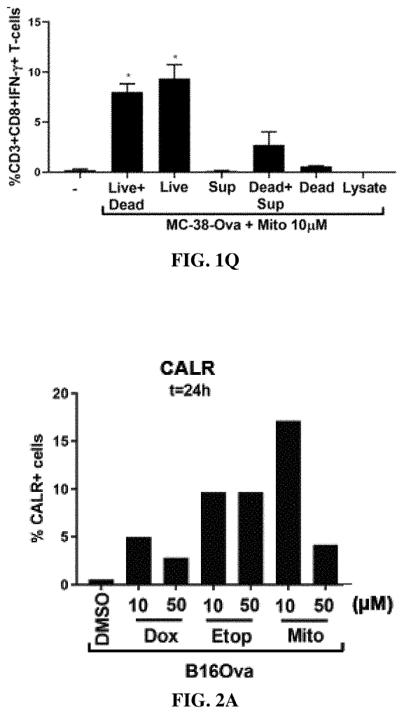

The isolated activated cells may be used in a two step ex vivo cross-presentation assay to establish the cells' ability to prime T cells. A diagram of the method is shown in FIG. 1A.

Step One

The assay in step one typically includes the isolated activated cells and autologous or allogeneic APCs, such as mononuclear cells or dendritic cells, co-cultured together. The cells are co-cultured together for a period of at least 3 hours, but typically between about 3 hours and 48 hours, such as 24 hours. The ratio of the isolated activated cells to APCs may be 10:1, 9:1, 8:1, 7:1, 6:1, 5:1, 4:1, 3:1, 2:1, 1:1, or 0.5:1, but typically is between about 8:1 and 2:1, such as 4:1.

The co-culture is then washed and the APCs are taken into step two of the assay.

Step 2

The assay in step two typically includes the APCs from step one and autologous or allogeneic CD3+CD8+ T cells co-cultured together. The cells are co-cultured together for a period of at least 3 hours, but typically between about 3 hours and 48 hours, such as 12-15 hours. The ratio of the APCs to T cells may be 5:1, 4:1, 3:1, 2:1, 1:1, or 0.5:1, typically is between about 4:1 and 0.5:1, such as 2:1.

The assay typically includes a control condition where step one does not include isolated activated cells, but instead includes untreated cells as controls. The control may also be isolated cells treated with a drug that is known not be genotoxic and/or not to induce immunogenic cell death markers in the cells.

The co-cultures are then subjected to intracellular cytokine staining for IFNγ and then analyzed by flow cytometry to identify the percentage of CD3+CD8+IFNγ+ T cells.

The increase in percentage of CD3+CD8+IFNγ+ T cells may be detected when activated cells are co-cultured with dendritic cells and CD8+ T cells, and the percentage of CD3+CD8+IFNγ+ T cells is measured by flow cytometry. The percentage of CD3+CD8+IFNγ+ T cells is then compared to the percentage of CD3+CD8+IFNγ+ T cells in a control co-culture of dendritic cells and CD8+ T cells in the absence of the isolated activated cells, the two co-cultures having similar or the same treatment and cell numbers.

The increase may be an increase by at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fold over the values detected in the control cells or in the control co-culture.

### IV. Methods of Using the Cellular Vaccines

Typically, the cellular vaccines are used in subjects with cancer to activate cytotoxic immune responses against the cancer cells in vivo, provide tumor regression, and enhances survival from cancer. The vaccines may also prevent tumor recurrence, for example, for a period of about 5 years to about 10 years, such as for at least 5 years, for at least 6 years, for at least 7 years, for at least 8 years, for at least 9 years, or for at least 10 years. Additionally, or alternatively, the vaccines may induce a long-lasting anti-tumor immunological memory.

Typically, the cellular vaccine is administered into a subject's tumor, i.e., intratumorally. The administration may be repeated as needed. The subject may then be followed for the state of tumor regression and changes in the circulating CD3+CD8+IFNγ+ T cells.

In some embodiments, the cellular vaccine is administered in combination with an ICI, for example, one or more of those provided above. Preferably, the ICI is administered to the subject systemically. Additionally, or alternatively, the ICI can be administered locally, for example intratumorally. The ICI can be administered together or separately from the isolated, activated tumor cells. The ICI can be form part of the cellular vaccine composition itself, and can be a separate composition. The ICI can be administered before, along with, after, or any combination thereof, the isolated, activated tumor cells. In preferred embodiments, the ICI is administered systemically, while the cells are administered intratumorally. In some embodiments, the ICI is administered before the administration of the tumor cell vaccine. The ICI may be administered 48 hours, 36 hours, 24 hours, 12 hours, or 6 hours before the administration of the tumor cell vaccine. Preferably, the ICI is administered 24 hours before the administration of the tumor cell vaccine.

A. Subjects to be Treated

Typically, the subjects to be treated have a proliferative disease, such as a benign or malignant tumor. In some embodiments, the subjects to be treated have been diagnosed with stage I, stage II, stage III, or stage IV cancer. The subjects may be in remission from cancer.

Examples of cancers to be treated include, but are not limited to Leukemia, AIDS-Related Cancers Kaposi Sarcoma, AIDS-Related Lymphoma, Lymphoma, Astrocytomas, Basal Cell Carcinoma, Bile Duct Cancer, Bladder Cancer, Bone, Brain Tumors, Breast Cancer, Bronchial Tumors, Burkitt Lymphoma, Cardiac (Heart) Tumors, Cervical Cancer, Chronic Myeloproliferative Neoplasms, Colorectal Cancer, Craniopharyngioma, Embryonal Tumors, Endometrial Cancer, Ependymoma, Esophageal, Esthesioneuroblastoma, Eye Cancer Intraocular Melanoma, Retinoblastoma, Fallopian Tube Cancer, Fibrous Histiocytoma of Bone, Gallbladder Cancer, Gastric (Stomach) Cancer, Gastrointestinal Carcinoid Tumor, Head and Neck Cancer, Hepatocellular (Liver) Cancer, Hodgkin Lymphoma, Intraocular Melanoma, Pancreatic Neuroendocrine Tumors, Kaposi Sarcoma, Langerhans Cell Histiocytosis, Lip and Oral Cavity Cancer, Liver Cancer (Primary), Lung Cancer, Lymphoma, Melanoma, Mesothelioma, Non-Hodgkin Lymphoma, Non-Small Cell Lung Cancer, Osteosarcoma and Malignant Fibrous Histiocytoma of Bone, Ovarian Cancer, Pancreatic Cancer and Pancreatic Neuroendocrine Tumors (Islet Cell Tumors), Pregnancy and Breast Cancer, Osteosarcoma, Rhabdomyosarcoma, Uterine Sarcoma, Vascular Tumors, Skin Cancer, Small Cell Lung Cancer, Small Intestine Cancer, Soft Tissue Sarcoma, Squamous Cell Carcinoma, Squamous Neck Cancer with Occult Primary, T-Cell Lymphoma, Testicular Cancer, Throat Cancer, Thymoma and Thymic Carcinoma, Thyroid Cancer, Thyroid Tumors, Uterine Cancer, Endometrial and Uterine Sarcoma, and Vaginal Cancer.

1. Tumor Regression

The cellular vaccine typically provides an anti-tumor immunological reaction resulting in tumor size regression. The cellular vaccines may reduce the tumor size of individual tumors. The cellular vaccines may also reduce the number of tumors in a subject. Generally, the cellular vaccines may reduce the tumor size and/or the number of tumors by about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% from the initial tumor size or tumor number.

The tumor size and tumor number may be monitored with the methods routinely used in oncology. The methods used to detect reduction in tumor size or cancers in remission include biopsies, non-invasive imaging methods, recording methods, laboratory tests detecting blood biomarkers, and/or visual evaluation.

Examples of non-invasive methods include contrast-enhanced and non-enhanced magnetic resonance imaging (MRI), computerized tomography (CT), positron-emission tomography (PET), single-photon emission computed tomography (SPECT), X-ray, mammography, ultrasonography or ultrasound, endoscopy, elastography, tactile imaging, thermography, and medical photography.

Other methods include measurement and recording techniques, such as electroencephalography (EEG), magnetoencephalography (MEG), and electrocardiography (ECG).

Examples of laboratory tests include complete blood count (CBC), blood protein testing (electrophoresis), tumor marker tests, and detecting circulating tumor cells circulating CD3+CD8+IFNγ+ T cells.

2. Reduced Tumor Recurrence

In some aspects, the subjects receiving the cellular vaccine may have prolonged disease-free survival from the cancer than what is a typical prognosis for the disease. Prognosis may include estimating cancer-specific survival (the percentage of patients with a specific type and stage of cancer who have not died from their cancer during a certain period of time after diagnosis), relative survival (the percentage of cancer patients who have survived for a certain period of time after diagnosis compared to people who do not have cancer), overall survival (the percentage of people with a specific type and stage of cancer who have not died from any cause during a certain period of time after diagnosis), or disease-free survival (also referred to as recurrence-free or progression-free survival, is the percentage of patients who have no signs of cancer during a certain period of time after treatment). Prognosis may also include a negative prognosis for positive outcome, or a positive prognosis for a positive outcome.

Good prognosis, or positive prognosis, indicates that the subject is expected (e.g. predicted) to survive and/or have no, or is at low risk of having, recurrence or distant metastases within a set time period. The term “low” is a relative term. A “low” risk can be considered as a risk lower than the average risk for a heterogeneous cancer patient population. A “low” risk of recurrence may be considered to be lower than 5%, 10%, or 15% the average risk for an heterogeneous cancer patient population. The risk will also vary in function of the time period. The time period can be, for example, five years, ten years, fifteen years or even twenty years after initial diagnosis of cancer or after the prognosis was made.

Generally, subjects receiving the cellular vaccine have an increased median survival, which refers to the length of time from either the date of diagnosis or the start of treatment for a disease, such as cancer, during which half of the patients in a group of patients diagnosed with the disease are still alive.

3. Long-Lasting Anti-Tumor Immunological Memory

The cellular vaccines provide cytotoxic immune response against the cancer cells of the subject. The vaccines also provide tumor regression when injected intratumorally, and enhance survival from cancer. Additionally or alternatively, the vaccines prevent tumor recurrence and induce a long-lasting anti-tumor immunological memory.

The “immune response” refers to responses that induce, increase, or perpetuate the activation or efficiency of innate or adaptive immunity.

The immune response can be induced, increased, or enhanced by the vaccine as compared to a control. For example an immune response in a subject may be induced, increased, or enhanced by the vaccine delivered intratumorally, as compared to the immune response in a control subject who did not receive the vaccine, or the vaccine in the control subject was delivered to an alternative delivery site. Typically, the vaccines enhance activation of cancer-specific T cells (i.e., increase antigen-specific proliferation of T cells, enhance cytokine production by T cells, stimulate differentiation ad effector functions of T cells and/or promote T cell survival) or overcome T cell exhaustion and/or anergy, as compared to the control.

The cellular vaccines can provide an improved effector cell response, including a higher effector cell response such as a CD8 or CD4 response obtained in a patient after administration of the vaccine composition than that obtained after administration of the same composition without the isolated activated cells.

In a preferred embodiment, the vaccine increases the number of CD3+CD8+ T cells producing IFN-gamma, and/or increases the production of IFN-gamma in the existing CD3+CD8+ T cells.

In some embodiments, the administration of the vaccine alternatively or additionally induces an improved B-memory cell response in patients administered the vaccine compared to a control. An improved B-memory cell response is intended to mean an increased frequency of peripheral blood B lymphocytes capable of differentiation into antibody-secreting plasma cells upon antigen encounter as measured by stimulation of ex vivo differentiation.

B. Administering the Vaccine

The cellular vaccines are typically administered intratumorally in cancers with solid tumors. Additionally or alternatively, the cellular vaccines may be administered locally or systemically to induce immune responses against cancers, particularly when there are no visible or detectable solid tumors, such as in patients in remission, or in patients with leukemia.

Typically administration is injection or infusion of a single injection dose. The administration may be repeated as many times as is necessary to establish an anti-tumor immune effector reactions and/or a long-lasting anti-tumor immunological memory.

1. Dosage

Typically, a single vaccine contains between about 10⁴ and 10⁸ isolated and activated cells for a single injection dose. The vaccine may also include autologous or allogeneic APCs at between about 10⁴ and 10⁸ cells per injection dose. The vaccine may also include autologous or allogeneic T cells at between about between about 10⁴ and 10⁸ cells per injection dose.

Typically, the vaccine contains between about 10⁴ and 10⁹ isolated and activated cells per injection dose. Generally, the vaccine may contain any number of isolated activated cells in this range, such as about 10⁴, about 10⁵, about 10⁶, about 10⁷, about 10⁸, or about 10⁹ cells. Preferred ranges include between about 10⁴ and 10⁷, such as between about 10⁴ and 1×10⁶, between about 10⁴ and 2×10⁶, between about 10⁴ and 3×10⁶, between about 10⁴ and 4×10⁶, between about 10⁴ and 5×10⁶, between about 10⁴ and 6×10⁶, between about 10⁴ and 7×10⁶, between about 10⁴ and 8×10⁶, between about 10⁴ and 9×10⁶, between about 10⁴ and 10×10⁶ isolated activated cells per injection dose.

If APCs are present, the ratio of the isolated activated cells to APCs may be 10:1, 9:1, 8:1, 7:1, 6:1, 5:1, 4:1, 3:1, 2:1, 1:1, or 0.5:1, but typically is between about 8:1 and 2:1, such as 4:1. If present, the APCs and T cells may be present at an APCs to T cell ratio of 5:1, 4:1, 3:1, 2:1, 1:1, or 0.5:1, preferably between about 4:1 and 0.5:1, such as 2:1.

The vaccines may also include ICI between about 0.1 mg/kg and about 100 mg/kg of the body weight of the patient in a single injection dose. Suitable amounts of the ICI in the vaccine include between about 0.1 mg/kg and about 500 mg/kg, between about 0.1 mg/kg and about 250 mg/kg, between about 0.1 mg/kg and about 100 mg/kg, between about 0.1 mg/kg and about 80 mg/kg, and between about 0.1 mg/kg and about 60 mg/kg. Specific concentrations of the ICI include 0.1 mg/kg, 0.2 mg/kg, 0.3 mg/kg 0.4 μM, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, and 50 mg/kg.

Dosing is dependent on severity and responsiveness of the disease condition to be treated, with the course of treatment lasting from several days to several months, or until reduction in tumor size (such as tumor area or tumor volume), tumor number, or one or more symptoms of the disease are observed. Persons of ordinary skill can determine optimum dosages, dosing methodologies and repetition rates, which may vary depending on the relative potency of individual vaccines, and can generally be estimated based on EC50s found to be effective in ex vivo assay and in in vivo animal models.

In some embodiments, the effect of the treatment is compared to a conventional treatment that is known the art.

### V. Kits

The cellular vaccines described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits with one or more dosages packed for injection into a subject, and may include a pre-measured dosage of the vaccine in a sterile needle, ampule, tube, container, or other suitable vessel.

The kits may include instructions for dosages and dosing regimens. The kits may also contain combinations of pharmaceutical compositions, such as ICI, for co-administration.

The disclosed compositions and methods of use thereof can be further understood through the following numbered paragraphs.

1. A composition for treating a patient with cancer, and/or preventing recurrence of the cancer, the composition comprising isolated, activated, primary tumor cells.

2. The composition of paragraph 1, wherein the cells are live, injured cells.

3. The composition of paragraphs 1 or 2, wherein the isolated activated cells are activated with one or more genotoxic drugs selected from the group consisting of alkylating agents, antimetabolites, antimitotics, anthracyclines, cytotoxic antibiotics, and topoisomerase inhibitors, and, optionally, with one or more MAPK-activated protein kinase-2 (MK2) inhibitors.

4. The composition of any one of paragraphs 1-3, wherein the genotoxic drug is selected from the group consisting of doxorubucin, etoposide, mitoxantrone, cisplatin, oxaliplatin, 5-fluorouracil, paclitaxel, irinotecan, camptothecin, and cyclophosphamide.

5. The composition of any one of paragraphs 1-4, wherein the cells are activated with one or more genotoxic drug(s) at a concentration between about 0.1 μM and 1000 μM.

6. The composition of paragraph 5, wherein the concentration of drug is sufficient to injure the cells and induce stress signaling, but not sufficient to induce maximal cell death of the cells.

7. The composition of any one of paragraphs 1-6, wherein the isolated activated tumor cells comprise cells with DNA damage, growth arrest, and/or necroptosis.

8. The composition of any one of paragraphs 1-7, wherein at least 1% of the isolated activated tumor cells comprise cells with necroptosis, as measured by flow cytometry.

9. The composition of any one of paragraphs 1-8, wherein the isolated activated tumor cells comprise between 1% and 100% cells, such as i) at least about 5%, ii) at least about 7.5%, iii) at least about 10%, or at least about 12% of the cells with externalized calreticulin, as detected by flow cytometry.

10. The composition of any one of paragraphs 1-9, wherein the isolated activated tumor cells comprise a) cells with between 1 fold and 15 fold greater, such as i) at least about 1.5 fold, ii) at least about 2 fold, iii) at least about 3 fold, or at least about 5 fold greater activated receptor-interacting protein kinase 1 (RIPK1), optionally as determined by Western blotting; b) cells with activated NF-κB; c) or a combination of a) and b).

11. The composition of any one of paragraphs 1-10, wherein the cells comprise intact, induced, or increased DNA damage signaling.

12. The composition of paragraph 11, wherein the DNA damage signaling comprises phosphorylation of one or more substrates of protein kinase ataxia-telangiectasia mutated (ATM), serine/threonine-protein kinase ATR, or a combination thereof.

13. The composition of any one of paragraphs 1-12, wherein the cells comprise induced or increased phosphorylation of p38MAPK.

14. The composition of any one of paragraphs 1-13, wherein the cells are free from in vitro or ex vivo transformation or transfection of a heterologous nucleic acid expression construct.

15. The composition of any one of paragraphs 1-13, wherein the cells are in vitro or ex vivo transformed or transfected with a heterologous nucleic acid expression construct.

16. The composition of paragraph 15, wherein the heterologous nucleic acid expression construct is for expression of one or more cytokines and/or signaling molecules, preferably wherein the cytokines and/or signaling molecules are downstream of RIPK1 and NF-kB, optionally wherein at least one of the cytokines is GM-CSF.

17. The composition of any one of paragraphs 1-16, further comprising dendritic cells, and/or T cells.

18. The composition of paragraph 17, wherein the dendritic cells and/or the T cells are autologous or allogenic.

19. The composition of any one of paragraphs 1-18, wherein tumor cells are cells from a breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, maxillary sinus cancer, bladder cancer, esophageal cancer, Hodgkin's disease, head and neck cancer, or adrenocortical cancer.

20. The composition of any one of paragraphs 1-19, comprising one or more immune checkpoint inhibitors (ICI).

21. The composition of paragraph 20, wherein the ICI is a small molecule, antibody, or antibody fragment against a molecule selected from the group consisting of programmed cell death protein 1 (PD-1), PD-1 Ligand 1 (PD-L1), and cytotoxic T-lymphocyte-associated antigen 4 (CTLA-4).

22. The composition of paragraph 20 or 21, wherein the ICI is selected from the group consisting of nivolumab, pembrolizumab, atezolizumab, avelumab, durvalumab, cemiplimab, CT-011, vopratelimab, danvatirsen, cetrelimab, and ipilimumab.

23. The composition of any one of paragraphs 20-22, wherein the ICI is at a dose between about 0.1 mg/kg and about 100 mg/kg of the body weight of the patient.

24. The composition of any one of paragraphs 1-23, in a formulation and dosage suitable for intratumoral injection.

25. The composition of any one of paragraphs 1-24, wherein the isolated activated tumor cells are isolated from a patient's tumor.

26. A composition for treating a patient with cancer, and/or preventing recurrence of the cancer, the composition comprising live, isolated, primary tumor cells activated by contacting the cells with an effective amount of a genotoxic drug to injure the cells and induce stress signaling, but not sufficient to induce maximal cell death of the cells.

27. The composition of paragraph 26, wherein the stress signaling comprises a DNA damage signaling pathway.

28. The composition of paragraph 27, wherein the stress signaling pathway comprises protein kinase ataxia-telangiectasia mutated (ATM), serine/threonine-protein kinase ATR, or a combination thereof.

29. The composition of paragraphs 26 or 27, wherein the cells comprise induced or increased phosphorylation of p38MAPK.

30. The composition of paragraphs 27 or 28, wherein the genotoxic drug is doxorubucin, etoposide, or mitoxantrone.

31. A method of treating a patient with cancer, and/or preventing recurrence of the cancer, comprising administering to the patient an effective amount of the composition of any one of paragraphs 1-30.

32. The method of paragraph 31, wherein the composition is administered by intratumoral injection.

33. The method of paragraph 31 or 32, comprising administering an effective amount of one or more immune checkpoint inhibitor(s) (ICI).

34. The method of paragraph 33, wherein the ICI is administered before, during, or after administering the composition.

35. The method of any one of paragraphs 31-34, wherein the composition comprises between about 10⁴ and about 10⁹ isolated activated tumor cells activated with an effective amount of one or more genotoxic drug(s), optionally treated with one or more MAPK-activated protein kinase-2 (MK2) inhibitors.

36. The method of any one of paragraphs 31-35, wherein the composition comprises tumor cells isolated from a tumor of the patient.

37. The method of any one of paragraphs 31-36, comprising, prior to administering the cells to the subject, screening genotoxic drugs on samples from isolated tumor cells and selecting a genotoxic drug that induces at least 1% necroptosis in the sample, optionally as measured by flow cytometry, and treating the cells with the selected drug ex vivo.

38. The method of any one of paragraphs 31-37, comprising, prior to administering the cells to the subject, screening genotoxic drugs on samples from isolated tumor cells and identifying the genotoxic drug that activates receptor-interacting protein kinase 1 (RIPK1) and/or activates or does not substantially inhibit NF-κB in the sample, optionally as measured by Western blotting and/or flow cytometry, and treating the cells with the selected drug ex vivo.

39. An ex vivo assay for personalized treatment of a patient with cancer, the assay comprising:

treating a plurality of samples of tumor cells isolated from the patient with genotoxic drugs to produce activated cells, and

selecting a drug and/or dosage or concentration thereof that produces activated tumor cells with the increased immunogenic potential as the drug for the personalized treatment of the patient with cancer, optionally wherein the drug produces activated tumor cells with the highest immunogenic potential of the tested drugs.

40. The assay of paragraph 39, wherein each sample of the isolated tumor cells is treated with a single genotoxic drug.

41. The assay of paragraph 39 or 40, wherein the genotoxic drug is at a concentration between about 0.1 μM and about 1000 μM.

42. The assay of paragraph 41, wherein the cells are contacted with different amounts of the genotoxic drug to identify a dosage or concentration that injures the cells and induces stress signaling, but is not sufficient to induce maximal cell death of the cells.

43. The assay of paragraph 42, wherein the stress signaling comprises a DNA damage signaling pathway.

44. The assay of any one of paragraphs 39-43, wherein identifying is by detecting at least 1% necroptosis in the activated tumor cells, as measured by flow cytometry.

45. The assay of any one of paragraphs 39-44, wherein identifying is by detecting activated receptor-interacting protein kinase 1 (RIPK1), NF-κB, or combination thereof in the activated tumor cells, optionally as measured by Western blotting and/or flow cytometry.

46. The assay of any one of paragraphs 39-45, wherein the assay further comprises co-culturing the produced activated cells with patient's dendritic cells.

47. The assay of any one of paragraphs 39-46, wherein the assay further comprises co-culturing the produced activated cells with patient's dendritic cells and patient's T cells.

48. The assay of any one of paragraphs 39-47, comprising testing the produced activated tumor cells for improved dendritic-cell mediated T-cell priming.

49. The assay of any one of paragraphs 39-48, wherein activated cells with increased immunogenic potential comprise cells that induce an increase in the percentage of interferon (IFN)-gamma-producing cytotoxic T cells when the activated cells are co-cultured with patient's dendritic cells and patient's T-cells, and the highest immunogenic potential comprise cells that induce the greatest percentage of interferon (IFN)-gamma-producing cytotoxic T cells when the activated cells are co-cultured with patient's dendritic cells and patient's T-cells.

50. The assay of paragraph 49, wherein the percentage of IFN-gamma-producing cytotoxic T cells is measured by flow cytometry.

51. A personalized treatment of a patient with cancer, comprising administering into a tumor of the patient an effective amount of the patient's own activated tumor cells having an increased immunogenic potential, and optionally the highest immunogenic potential, as prepared according to the assay of any one of paragraphs 39-50.

52. The personalized treatment of paragraph 51, wherein the effective amount of the patient's own activated tumor cells comprises an amount between about 10⁴ and about 10⁹ cells activated tumor cells.

53. The personalized treatment of paragraph 51 or 52, further comprising administering the patient an effective amount of one or more immune checkpoint inhibitors (ICI).

54. The personalized treatment of paragraph 53, wherein the effective amount of one or more ICI is between about 0.1 mg/kg and about 100 mg/kg of the body weight of the patient.

55. The personalized treatment of paragraph 53 or 54, wherein the ICI is administered before, during, or after administering the patient's own activated tumor cells.

56. The personalized treatment of any one of paragraphs 51-55, wherein the ICI is a small molecule or antibody or antibody fragment against a molecule selected from the group consisting of programmed cell death protein 1 (PD-1), against PD-1 Ligand 1 (PD-L1), and against cytotoxic T-lymphocyte-associated antigen 4 (CTLA-4).

57. The personalized treatment of any one of paragraphs 51-56, wherein the ICI is selected from the group consisting of nivolumab, pembrolizumab, atezolizumab, avelumab, durvalumab, cemiplimab, CT-011, vopratelimab, danvatirsen, cetrelimab, and ipilimumab.

The present invention will be further understood by reference to the following non-limiting examples.

### EXAMPLES

Based in-part on an interest in cross-talk between the DNA damage response and signaling pathways that mediate tumor cell survival, apoptotic cell death, and innate immune activation (Cannell, et al., Cancer Cell., 28(5):623-637 (2015); Suarez-Lopez et al., Proc Natl Acad Sci USA., 115(18):E4236-E4244 (2018); Morandell, et al., Cell Rep., 5(4):868-77 (2013); Reinhardt, et al., Mol Cell, 40: 34-49 (2010); Floyd, et al., Nature, 498:246-250 (2013); Hsu, et al., Shock, 44(2):128-36 (2015)), experiments were designed to investigate whether signaling pathways activated in response to specific types of DNA damaging chemotherapy could enhance subsequent anti-tumor immune responses.

While the ability of specific chemotherapeutic compounds to enhance cross presentation of tumor antigens by dendritic cells has been characterized as “immunogenic cell death” (Obeid, et al., Nat Med., 13(1):54-61 (2007); Apetoh, et al., Nat Med., 13:1050-1059 (2007); Tesniere, et al., Oncogene, 29:482-491 (2010); Kepp, et al., OncoImmunology, 3(9):e955691 (2014)), the experiments below show that chemotherapy-induced cell stress signaling in live injured cells, but not the presence of dead cells, was the primary determinant of T-cell immunity. This effect seems to be mediated by RIPK1, p38MAPK and NF-kB signaling in the injured tumor cells. Furthermore, results show that direct intra-tumoral injection of ex vivo chemotherapy treated cells as an injured cell adjuvant, in combination with systemic ICI, but not systemic ICI alone, drives anti-tumor immunity and tumor regression in murine melanoma models.

### Example 1: Etoposide and Mitoxantrone-Treated Tumor Cells Induce DC-Mediated OT-I T-Cell Priming In Vitro

Materials and Methods

Reagents, Cell Lines and Mouse Strains

Mouse GM-CSF and AnnV-FITC were purchased from Biolegend. IL-4 was purchased from Thermo Fisher Scientific. Anti-CD3 (FITC) (145-2C11), Anti-CD8 (APC) (53-6.7), Anti-IFNγ (PE) (XMG1.2), Anti-CD45 (BUV395)(30-F11), Anti-CD24 (APC) (M1/69), Anti-Ly6C (BV605) (AL-21), Anti-F4/80 (BV711) (BM8), Anti-MHCII (PE-Cy7) (M5/14.15.2), Anti-CD11b (BV786) (M1/70), Anti-CD103 (BV421) (2E7) were purchased from ebioscience or Biolegend. H2-Kb/SIINFEKL (SEQ ID NO:1)-tetramer (PE-conjugated) was purchased from MBL Life Science. Necrostatin-1 and Z-VAD were purchased from Invivogen. Doxorubicin, Etoposide, Mitoxantrone, Cisplatin, Paclitaxel, Camptothecin, Irinotecan, 5-FU and cylcophosphamide were purchased from LC labs or Sigma. Oxaliplatin was purchased from Tocris Biosciences. An antibody against ovalbumin was purchased from Abcam (Cat #ab17293). PhosphoRIPK1 (S166) (Cat #31122S) and RIPK1 (Cat #3493T) antibodies were purchased from Cell Signaling Technology. Calreticulin antibodies were purchased from Invitrogen (Cat #PA3-900) and Cell Signaling Technology (Cat #12238T). CellTiter-Glo was purchased from Promega. CountBright absolute counting beads for flow cytometry, ACK lysis buffer, Lipofectamine RNAiMax transfection reagent, and LIVE/DEAD Fixable Aqua Dead Cell Stain kit were purchased from Thermo Fisher Scientific. HMGB1 ELISA kit was purchased from IBL international. CD8+ T-cell isolation kit was from STEM cell technologies. Anti-PD1 (clone RMP1-14) and anti-CTLA4 (clone 9D9) were from BioXcell. Anti-Batf3 antibody was purchased from Abcam (#ab211304).

B16F10 cells and MC-38 cells were obtained from ATCC. B16F10 cells were engineered to stably express ovalbumin (B16-Ova cells), as described previously (Moynihan K D., et al., Nat Med., 22(12):1402-1410 (2016)). MC-38 Ova cells were generated by transduction of MC-38 cells with pLVX-Ovalbumin-IRES-hygro, selection of stable expression clones using hygromycin, followed by isolation and expansion of single cell clones. Ovalbumin expression was verified by Western blotting. Calreticulin siRNA (silencer select ID #s63272) was purchased from Thermo Fisher Scientific. C57BL/6J WT, BATF3 (−/−), and OT-1 mice were purchased from Jackson laboratories.

BMDC Generation

Bone marrow was harvested from the femurs and tibias of Taconic C57BL/6 mice. The bone marrow was flushed out after nipping one end, and then centrifuged at 15,000×g for 15 s. Following 1 round of RBC lysis with ACK lysis buffer, cells were filtered through a 100 μm filter to remove aggregates, re-suspended at 1×10⁶ cells/ml, and cultured on a 10 cm bacterial plate (12 million cells per plate) in Iscove's Modified Dulbecco's Medium (IMDM) containing 10% FBS with antibiotics, 20 ng/ml each of GMCSF and IL-4 and 55 μM of b-mercaptoethanol. After 3 days, 75% of the media was replaced with fresh media containing growth factors. Dendritic cells, which were loosely adherent, were harvested by gentle pipetting on day 6 or 7 and used for the assay.

In Vitro Cross Presentation Assay

B16-Ova or MC-38 Ova cells were treated with various doses of chemotherapeutic drugs for 24 h followed by extensive washing in IMDM (10% FBS, P/S). Subsequently 1×10⁶ treated cells were co-cultured with 2.5×10⁵ BMDC per well of a 24-well plate for each condition tested. After 24 hours of co-culture, supernatants were removed from each well and the BMDC washed 2-3 times in T-cell media (RPMI containing 10% FBS, 20 mM HEPES, 1 mM sodium pyruvate, 55 uM b-mercaptoethanol, 2 mM L-glutamine, nonessential amino acids and antibiotics). CD8+OT-I T-cells isolated from spleens of OT-I mice were then co-cultured with the BMDC at 125,000 T-cells per well to achieve an effector to target ratio of 0.5. Where indicated, BMDC and/or T-cells were also exposed to chemotherapy drugs. After a 12-15 h incubation, IFN-g producing T-cells were identified and quantified by intra-cellular cytokine staining and flow cytometry using a BD LSR II or Fortessa flow cytometer. Cells were first gated for CD3 expression, then re-gated for CD8 and IFNg expression.

Cell Death and Viability Assays

For assessment of cell death, floating and attached cells were harvested after 48 hours of treatment with the indicated chemotherapeutic drugs. Attached cells were detached using 5 mM EDTA in PBS. The recovered cells were centrifuged at 250×g for 5 minutes, washed once in PBS containing 0.9 mM Ca2+ and 0.5 mM Mg2+ and then stained with AnnV-FITC for 15 minutes in Annexin binding buffer at room temperature according to the manufacturer's protocol (Biolegend). Cells were co-stained with DAPI at a final concentration of 1 μg/ml for 2 minutes in Annexin binding buffer, brought to a final volume of 500 ul using PBS containing 0.9 mM Ca2⁺ and 0.5 mM Mg2⁺ and analyzed by flow cytometry.

For assays of survival, 15,000 cells were plated per well in a 96-well plate in 100 μl media with 5 replicates per condition. Wells along the four edges of the plate were not used. Following cell attachment, the indicated drugs were added in an equal volume of media, and incubated for an additional 48 hours. The media was then removed and replaced with 100 ul of fresh media at room temperature. Following a 30 minute incubation at room temperature, 50 μl of CellTiter-Glo reagent was added, followed by 2 minutes of gentle mixing. The plate was incubated at room temperature for an additional 10 minutes. 100 μl of supernatant was transferred to a 96-well white opaque plate and luminescence was read on a Tecan microplate reader. Values were normalized to those of DMSO-treated control cells.

Statistics

All statistical analysis of data was performed using GraphPad Prism software. Comparisons of multiple experimental treatments to a single control condition were analyzed by ANOVA followed by Dunnett's multiple comparisons test. Comparisons between specific treatment groups were analyzed using a Student's t-test with Bonferroni correction for multiple hypothesis testing.

Results

To identify how DNA-damaging chemotherapy could be optimally used to enhance anti-tumor immune function, cytotoxicity assays were performed using the B16F10 melanoma tumor cell line expressing ovalbumin (B16-Ova). Cells were treated with clinically used chemotherapeutic agents followed by assaying for cell death by DAPI and Annexin V staining 48 hours later. It was observed that doxorubicin, etoposide, mitoxantrone, cisplatin, and 5-FU caused varying extents of apoptotic and non-apoptotic cell death, while oxaliplatin, cyclophosphamide, irinotecan, camptothecin and paclitaxel did not induce substantial amounts of cell death at the concentrations used at this time point. Irinotecan, oxaliplatin, and paclitaxel treatment, however, caused some degree of growth arrest. The results are shown in Tables 1 and 2.

TABLE 1
Percentage of cell death (shown as Mean and Range) from
treatment of B16F10 melanoma tumor cell line with the
indicated genotoxic agents and concentrations.

| | AnnV−DAPI+ | | AnnV+DAPI+ | | AnnV+DAPI− | | AnnV−DAPI− | |
|---|---|---|---|---|---|---|---|---|
| | (%) | | (%) | | (%) | | (%) | |
| Sample | Mean | Range | Mean | Range | Mean | Range | Mean | Range |
| — (Control) | 3.30 | 10.13 | 6.14 | 5.78 | 1.11 | 1.64 | 89.45 | 6.90 |
| Dox 10 uM | 0.04 | 0.03 | 47.95 | 0.90 | 31.60 | 1.00 | 20.40 | 2.00 |
| Dox 50 uM | 0.04 | 0.00 | 52.10 | 1.60 | 27.05 | 2.30 | 20.80 | 0.80 |
| E10 | 6.54 | −5.60 | 51.00 | 6.40 | 11.55 | 3.90 | 30.90 | 4.80 |
| E50 | 4.57 | 0.18 | 47.85 | 2.70 | 10.95 | 1.50 | 36.60 | 4.40 |
| M10 | 9.44 | 2.13 | 52.35 | 3.90 | 17.15 | 3.30 | 21.05 | 2.70 |
| M50 | 8.50 | 2.25 | 73.80 | 0.20 | 11.65 | 2.30 | 6.08 | 0.27 |
| Cis10 | 0.32 | 0.13 | 2.76 | 0.00 | 3.68 | 0.94 | 93.25 | 1.10 |
| Cis50 | 2.96 | 0.48 | 18.75 | 0.10 | 46.80 | 4.60 | 31.55 | 4.90 |
| Oxal10 | 1.11 | 1.76 | 1.73 | 0.74 | 2.65 | 4.66 | 94.50 | 4.80 |
| Oxal50 | 1.09 | 1.26 | 4.25 | 2.09 | 4.23 | 5.67 | 90.45 | 4.10 |
| 5-FU 10 | 1.23 | 0.15 | 5.29 | 0.13 | 9.50 | 0.55 | 84.00 | 0.80 |
| 5-FU 50 | 1.33 | 0.25 | 6.78 | 1.06 | 28.05 | 2.10 | 63.85 | 3.50 |
| Pac 200 nM | 2.18 | 0.44 | 4.79 | 0.83 | 7.20 | 0.47 | 85.85 | 1.70 |
| Pac 1 uM | 2.79 | 0.37 | 2.98 | 0.11 | 3.03 | 0.03 | 91.25 | 0.50 |
| Iri 50 nM | 1.87 | 0.09 | 3.79 | 1.22 | 0.36 | 0.01 | 93.95 | 1.30 |
| Iri 250 nM | 1.63 | 0.02 | 5.06 | 0.58 | 0.54 | 0.27 | 92.75 | 0.90 |
| Iri 1 uM | 0.89 | 0.04 | 6.38 | 4.37 | 1.74 | 0.36 | 91.00 | 4.80 |
| CPT 50 nM | 1.35 | 0.18 | 7.03 | 2.21 | 1.26 | 0.19 | 90.40 | 2.20 |
| CPT 250 nM | 0.58 | 0.23 | 4.71 | 0.69 | 4.35 | 0.46 | 90.35 | 0.50 |
| CPM 40 uM | 0.82 | 0.20 | 7.70 | 0.18 | 1.44 | 0.05 | 90.05 | 0.50 |
| CPM 200 uM | 0.88 | 0.27 | 10.99 | 3.82 | 1.09 | 0.44 | 87.00 | 4.60 |

TABLE 2
Percentage of cell viability (shown as Mean and Standard Error
of the Mean (SEM)) from treatment of B16F10 melanoma tumor cell
line with the indicated genotoxic agents and concentrations.

Cell viability (%)

| | MEAN | SEM |
|---|---|---|
| (Control) | 100 | 0 |
| Dox 10 uM | 0.504521 | 0.041578 |
| Dox 50 uM | 0.125918 | 0.005916 |
| Etop 10 uM | 34.91644 | 0.721056 |
| Etop 50 uM | 36.05653 | 0.985174 |
| Mito 10 uM | 0.074256 | 0.008849 |
| Mito 50 uM | 0.000452 | 0.00816 |
| Cis 10 uM | 53.38704 | 1.136774 |
| Cis 50 uM | 6.421317 | 0.227425 |
| Oxal 10 uM | 45.08813 | 1.405225 |
| Oxal 50 uM | 44.20638 | 1.805505 |
| 5-FU 10 uM | 48.97402 | 0.941243 |
| 5-FU 50 uM | 42.79094 | 0.743983 |
| Pac 0.2 uM | 69.79857 | 1.724866 |
| Pac 1 uM | 73.97099 | 1.110773 |
| IRI .05 uM | 96.79659 | 0.905893 |
| IRI 0.25 uM | 99.7587 | 1.022214 |
| IRI 1 uM | 97.91434 | 0.971338 |
| CPT .05 uM | 91.0775 | 0.647099 |
| CPT .25 uM | 77.56982 | 2.092905 |
| CPM 40 uM | 98.47008 | 1.170661 |
| CPM 200 uM | 100.7488 | 0.944119 |

An experimental system was developed to assess dendritic cell-mediated T-cell priming using the B16-Ova cells, in combination with primary bone-marrow derived dendritic cells (BMDC) and OT-I CD8+ T-cells expressing a TCR transgene that specifically recognizes the Ova-derived peptide SIINFEKL (SEQ ID NO:1) in the context of H2-Kb (OT-1) (Clarke, et al., Immunol Cell Biol., 78(2):110-7 (2000); Hogquist, et al., Cell, 76(1):17-27 (1994)). As shown in FIG. 1A, in this assay B16-Ova cells were treated with the subset of drugs that induced substantial cell death, the drug was washed out after 24 hours, and the treated tumor cells were added to BMDCs for an additional 24 hours. The treated B16-Ova cells/BMDC co-culture was then incubated with purified CD8+ T-cells obtained from the spleens of OT-1 mice, and the appearance of IFNg+CD8 T-cells was measured 12-15 hours later by intracellular staining and subsequent quantification using flow cytometry.

Treatment of B16-Ova cells with either 50 μM etoposide or 10 μM mitoxantrone, followed by co-culture with BMDC, proved highly effective at inducing the appearance of IFNγ+OT-1 CD8+ T-cells (FIGS. 1B-1C, 1E, and Table 3). Similar results were obtained using the murine colon carcinoma cell line MC-38 stably expressing ovalbumin (MC-38-Ova), although in this case a lower dose of etoposide (10 μM), proved to be the most effective treatment (FIG. 1D, and Table 4). The effectiveness of these DNA damaging drugs at inducing T-cell IFN-γ responses was highly dose-dependent for each cell line.

In contrast to etoposide and mitoxantrone, another topoisomerase-II inhibitor, doxorubicin, was ineffective at inducing DC-mediated IFN-γ in T-cells, despite causing similar or higher levels of total cell death (Table 3, Table 4, FIG. 1E).

TABLE 3
Percentage of CD3+CD8+IFN-γ T cells after
B16-Ova cells were treated with indicated genotoxic
agents and concentrations, followed by co-culture with BMDC.

% CD3+CD8+IFN-γ+ T-cells

| | MEAN | SEM |
|---|---|---|
| Unstimulated | 0.23 | 0.21 |
| DMSO | 0.04 | 0.02 |
| Dox 10 uM | 0.13 | 0.04 |
| Dox 50 uM | 0.28 | 0.21 |
| Etop 10 uM | 0.86 | 0.60 |
| Etop 50 uM | 4.80 | 0.35 |
| Mito 10 uM | 5.57 | 1.30 |
| Mito 50 uM | 0.44 | 0.25 |

TABLE 4
Percentage of CD3+CD8+IFN-γ T cells after
MC-38-Ova cells were treated with indicated genotoxic
agents and concentrations, followed by co-culture with BMDC.

| % CD3+CD8+IFN-γ+ T-cells | MEAN | SEM |
|---|---|---|
| Unstimulated | 0.067 | 0.007371 |
| DMSO | 0.709 | 0.019313 |
| Dox 10 uM | 1.217333 | 0.142667 |
| Dox 50 uM | 0.086667 | 0.029678 |
| Etop 10 uM | 13.13333 | 0.088192 |
| Etop 50 uM | 3.83 | 0.118462 |
| Mito 10 uM | 3.433333 | 0.38602 |
| Mito 50 uM | 0.148 | 0.148 |

### Example 2: Live Injured Cells, Rather than Dead Cells, are Determinants of DC-Mediated IFN-γ Induction in T-Cells in Response to Mitoxantrone and Etoposide Treatment

Materials and Methods

Fractionation of Live and Dead Fractions from Chemotherapy-Treated Cells

B16-Ova cells or MC-38-Ova cells were treated with various doses of chemotherapy as indicated in FIGS. 1F-1I for 24 hours after which the floating fraction of cells was transferred to a separate tube and washed with PBS (for AnnV/DAPI staining) or IMDM (for co-culture with BMDC). The attached fraction was rinsed 1× with PBS, detached using 5 mM EDTA (in PBS), washed with PBS or IMDM and transferred to a separate tube. Separately, cells treated with chemotherapy for 24 h were re-plated at 1 million cells per well of a 24-well plate in 500 ul of IMDM (10% FBS; P/S). Cell-free supernatants were collected after a further 24 h. As shown in FIGS. 1F-1I, staining with AnnV and DAPI of the attached and floating fractions after chemotherapy treatment and fractionation revealed that the attached fraction is predominantly AnnV and DAPI double negative indicating that the majority of cells in this fraction are live injured cells. On the other hand, the floating fraction (labeled as ‘suspension’ in FIGS. 1F-1I) consists of cells that predominantly stain positive for AnnV and/or DAPI indicating that the majority of cells in this fraction are dead cells. Lysate of the total chemotherapy-treated cell mixture was generated by three rounds of freeze-thawing by alternate incubations in liquid nitrogen and a 37 C water bath.

Results

As shown in Table 1 and 2, both drugs that effectively induced DC-mediated IFN-γ in CD8+ T-cells induced substantial amounts of apoptotic and non-apoptotic tumor cell death compared to drugs that failed to elicit an immune response, although notably, doxorubicin also caused similar amounts of cell death but was immunologically silent. Curiously, at the doses used in FIG. 1E and Tables 3 and 4, the specific doses of mitoxantrone and etoposide that were maximally effective were not the doses that caused the greatest amount of cell death. To investigate if the magnitude of T-cell IFN-γ responses directly correlated with the amount of dead cells present in the treated tumor cell fractions that were co-incubated with BMDC, tumor cells were treated with increasing doses of etoposide or mitoxantrone from 0 to 100 uM. As shown in FIG. 1J, B16-Ova cells treated with increasing doses of etoposide, induced a corresponding increase in the magnitude of IFN-γ responses in T-cells (using the assay described in FIG. 1A).

However, as shown in FIG. 1L, the proportion of dead cells (AnnV or DAPI single or double positive) present in the treated tumor cell mixture increases up to ˜30% at 25 uM etoposide, but stays unchanged (at ˜30%) between 25 and 75 uM and shows only a further small increase (by ˜5%) at 100 uM etoposide treatment. On the other hand, B16-Ova cells treated with 5 uM mitoxantrone induced the maximum IFN-γ responses in T-cells among the doses tested, while cells treated with 10 uM mitoxantrone induced a lower IFN-γ response which became undetectable at 25 uM and higher doses (FIG. 1K). The dead cell proportion in the mitoxantrone-treated B16-Ova cell mixture is equivalent between 5 and 10 uM (˜50%) and increases to greater than 90% at 25 uM and higher doses (FIG. 1J). Together these results indicate that the proportion of dead cells in both the etoposide and mitoxantrone-treated B16-Ova tumor cell mixtures does not correlate with the magnitude of T-cell IFN-γ responses induced.

Since the above results indicated that there was no direct positive correlation between the proportion of dead cells induced by etoposide or mitoxantrone treatment, and the DC-mediated IFN-γ responses in T-cells, the specific contribution of the dead and live fractions of tumor cells induced by chemotherapy-treatment was further investigated. The etoposide- and mitoxantrone-treated cell cultures were fractionated into either cell-free supernatants, supernatants containing dead (AnnV+ and/or DAPI+) cells, or a separate fraction containing only the live (AnnV and DAPI double negative) injured cells (see methods and FIGS. 1F-1I). As shown in FIGS. 1N and 1O, each fraction was then co-cultured with BMDCs for 24 hrs, followed by the addition of OT-1 CD8+ T-cells for an additional 12-15 hrs, as described above. Neither the cell-free supernatants, nor the supernatants containing the dead cells were capable of inducing DC-mediated T-cell IFN-γresponses. Similarly, lysates generated by subjecting the chemotherapy-treated total cell mixture to three rounds of freeze-thawing (between liquid nitrogen and 37 C), upon co-incubation with BMDC, failed to induce IFN-γ in T-cells. In marked contrast, the fraction containing the adherent live injured cells were the most effective at inducing the expression of IFNγ in OT-1 T-cells. Similar behavior was also noted in the MC-38-Ova cells (FIGS. 1P and 1Q).

### Example 3: Conventional Immunogenic Death Markers do not Predict the Immunogenicity of Etoposide-Treated B16-Ova Cells

Materials and methods

Measurement of Immunogenic Cell Death Markers

For measurement of calreticulin surface exposure, B16-Ova cells were treated for 24 hours with various chemotherapy drugs. All attached and floating cells were harvested and washed in staining buffer (PBS containing 0.5% BSA) and incubated with anti-calreticulin antibodies for 1 hour on ice. Cells were washed once in staining buffer and then incubated with secondary AF488-conjugated secondary antibody for 1 hour at room temperature, washed again, re-suspended in staining buffer and analyzed by flow cytometry.

For HMGB1 measurement in cell culture media, B16-Ova cells were treated for 24 hours with various chemotherapy drugs, media was collected, and floating cells removed by centrifugation at 250×g for 5 minutes. Cell-free cell culture media was then analyzed by ELISA for HMGB1 according to the manufacturer's protocol.

For measurement of ATP levels, cell-free culture media obtained as above was analyzed by CellTiter-Glo according to the manufacturer's protocol. Values were converted to ATP concentrations using a standard curve generated using pure ATP.

Calreticulin siRNA Experimental Method

B16-Ova cells were transfected with calreticulin or control siRNA (30 nM final concentration) using Lipofectamine RNAiMax according to the manufacturer's protocol. 48 hours post-transfection, cells were used for the in vitro cross-presentation assay.

In Vitro Cross Presentation Assay

The in vitro cross presentation assay was performed as described in Example 1. Where indicated, B16Ova cells were co-treated with 20 μM of Necrostatin-1 or Z-VAD and etoposide or mitoxantrone at the concentrations of 10 or 50 μM for 24 hours prior to performance of the assay.

Results

In the in vitro assay system, both mitoxantrone and etoposide were found to induce dendritic cell-dependent T-cell priming Mitoxantrone has been previously reported to promote strong immunogenic cell death in CT26 mouse colon cancer cells based on its ability to induce calreticulin exposure on the cell surface (Obeid M, et al., Nat Med., 13(1):54-61 (2007)). Externalized calreticulin, along with HMGB1 and ATP release, have been identified as canonical markers of immunogenic cell death (Kepp 0, et al., Oncoimmunology., 3(9):e955691 (2014)). The finding that etoposide-treatment induced equivalent levels of IFNg+CD8+ T-cells as mitoxantrone in the in vitro assay for DC-mediated T-cell priming was unanticipated, as etoposide has been previously reported not to cause immunogenic cell death, and has been shown to be ineffective at inducing ER stress and calreticulin exposure in CT26 cells (Obeid M, et al., Nat Med., 13(1):54-61 (2007)).

To further examine this, B16-Ova cells were treated with etoposide, mitoxantrone or doxorubicin, and calreticulin exposure on the cell surface was measured at 24 hours. HMGB1 and ATP release during the first 24 hours of chemotherapy treatment, and during the 24-48 hours post-treatment window when the cells were co-cultured with BMDC was also analyzed (FIG. 1A). In previous reports, etoposide was not considered an immunogenic cell death inducing drug due to its inability to induce ER stress and calreticulin exposure in CT26 cells (Obeid, et al., Nat Med., 13(1):54-61 (2007)), despite inducing the release of HMGB1 and ATP (Bezu, et al., Frontiers in Immunology, 6:187. doi: 10.3389/fimmu.2015.00187. eCollection (2015)). However, etoposide was included in these experiments because it induced equivalent levels of IFN-γ+ CD8+ T-cells as mitoxantrone in the in vitro assay for DC-mediated T-cell responses. Doxorubicin was specifically chosen for comparison because it also belongs to the same class of DNA-damaging topoisomerase II inhibitors as etoposide and mitoxantrone, but did not induce T-cell priming in the assay system, although it has been reported to induce calreticulin exposure in CT26 cells (Obeid M, et al., Nat Med., 13(1):54-61 (2007), Bezu, et al., Frontiers in Immunology, 6:187. doi: 10.3389/fimmu.2015.00187. eCollection (2015)).

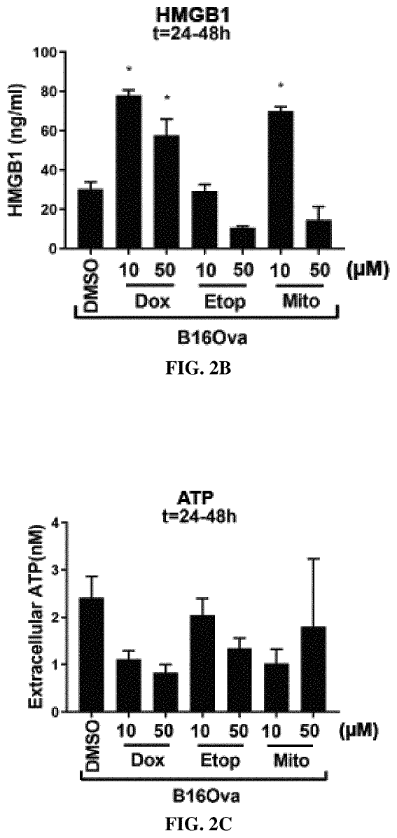

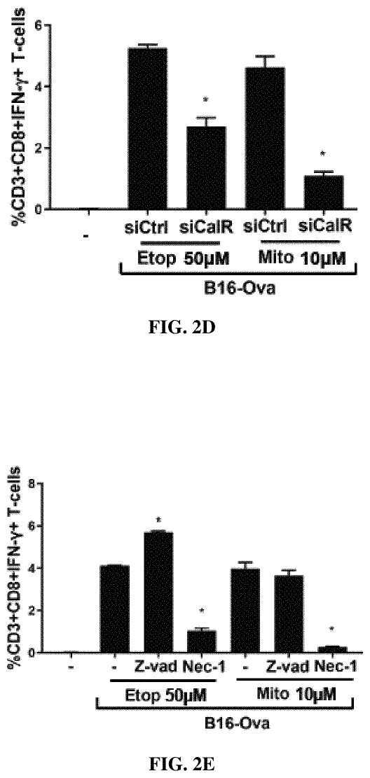

As shown in FIG. 2A, using two different anti-calreticulin antibodies (only one is shown), all drugs elicited only low levels of calreticulin exposure at this time point (24 hours), with <20% of the cells staining positively. Cells treated with mitoxantrone showed the highest level of externalized calreticulin when analyzed by flow cytometry after 24 hours of drug exposure. Cells treated with low or high etoposide concentrations showed intermediate levels of calreticulin exposure, while doxorubicin-treated cells showed the lowest levels. Cells treated with etoposide showed the lowest levels of HMGB1 release into the media during the 24-48 hours post-treatment window (FIG. 2B), despite being highly immunogenic. In contrast, doxorubicin treatment led to high levels of HMGB1 release, similar to what was observed with mitoxantrone (10 μM) treatment, despite its inability to promote BMDC-mediated T-cell priming. Similar HMGB1 release trends were observed in the first 24 hours of treatment. Substantial ATP release was detected after 24 hours of treatment in response to doxorubicin and mitoxantrone (FIG. 2C), which subsided by 48 hours. These results are summarized in Table 5.

TABLE 5
Values for the data presented in FIGS. 2A-2C

| | CALR | HMGB1 (ng/ml) | | ATP (nM) | |
|---|---|---|---|---|---|
| | (%) | MEAN | SEM | MEAN | SEM |
| DMSO | 0.55 | 30.49 | 3.47 | 2.41 | 0.45 |
| Dox 10 uM | 4.97 | 77.83 | 2.85 | 1.11 | 0.18 |
| Dox 50 uM | 2.82 | 57.75 | 8.18 | 0.83 | 0.17 |
| Etop 10 uM | 9.69 | 29.24 | 3.48 | 2.05 | 0.34 |
| Etop 50 uM | 9.69 | 10.65 | 0.84 | 1.35 | 0.22 |
| Mito 10 uM | 17.10 | 70.09 | 2.09 | 1.02 | 0.30 |
| Mito 50 uM | 4.15 | 14.63 | 6.75 | 1.80 | 1.43 |

To directly evaluate the contribution of calreticulin to DC-mediated T-cell priming in the assay, the experiments outlined in FIG. 1A were repeated following siRNA knockdown of calreticulin in B16-Ova cells. As shown in FIG. 2D and Table 6, siRNA knockdown of calreticulin prior to mitoxantrone treatment reduced the percentage of IFNg+ T-cells by ˜80%. By contrast, in response to etoposide treatment, calreticulin knock-down only reduced the percentage of IFNg+ T-cells by ˜50% compared to siRNA controls. These data show that the mechanism(s) of BMDC-mediated T-cell priming by etoposide-treated B16-Ova cells are only partially dependent on calreticulin externalization, and indicate that the canonical markers of immunogenic cell death (calreticulin, HMGB1, ATP) were unable to predict the ability of etoposide to act as an immune-activating drug in the assay.

TABLE 6
Percentage of CD3+CD8+IFN-γ+ T cells
following siRNA knockdown of CalR in B16-Ova cells
and treatment with the indicted genotoxic drugs
and concentrations prior to co-culture with BMDC.

% CD3+CD8+IFN-γ+ T-cells

| | MEAN | SEM |
|---|---|---|
| Unstimulated | 0.02 | 0.00 |
| siCtrl + Etop 50 uM | 5.25 | 0.12 |
| siCalR + Etop 50 uM | 2.71 | 0.28 |
| siCtrl + Mitro 10 uM | 4.63 | 0.36 |
| siCalR + Mito 10 uM | 1.10 | 0.13 |

While the levels of calreticulin exposure and HMGB1 release that was observed following exposure of B16-Ova cells to genotoxic drugs fits well with the ability of mitoxantrone to induce DC-mediated T-cell priming (Menger L., et al., Sci Transl Med., 4(143):143ra99 (2012)), these markers do not explain the comparable ability of etoposide treatment to induce DC-mediated IFN-g production in T-cells. To examine the contributions made by different signaling pathways that modulate terminal responses to genotoxic stress, either RIPK1 (shown to be a determinant of necroptosis), or caspases, (known determinants of apoptosis and pyroptosis) were next inhibited. Specifically, RIPK1 (a known determinant of necroptosis) (Silke, et al., Nature Immunology, 16:689-697 (2015)), caspases, (known determinants of apoptosis and pyroptosis) (Li and Yuan, Oncogene, 27:6194-6206 (2008)), NF-kB signaling (a critical regulatory node for survival and cytokine production) (Liu, et al., Signal Transduct Target Ther., 2017; 2:17023. doi: 10.1038/sigtrans.2017.23 (2017)) or p38MAPK (a well known master regulator of stress signaling, including those downstream of DNA-damage) Obata, et al., Crit Care Med., 28(4 Suppl):N67-77 (2000)) were inhibited.

B16-Ova cells were co-treated with etoposide or mitoxantrone in combination with the RIPK1 inhibitor necrostatin-1 (Nec-1), the pan-caspase inhibitor Z-VAD, the NF-kB signaling inhibitor Bay11-7085 (Pierce, et al., J Biol Chem., 272(34):21096-103. doi: 10.1074/jbc.272.34.21096 (1997)) or the p38MAPK inhibitor SB202190 (Davies, et al., Biochem J., 351(Pt 1): 95-105 (2000)), prior to co-culture with BMDC. As shown in FIG. 2E and Table 7, co-treatment with necrostatin-1 inhibited the ability of both etoposide and mitoxantrone-treated B16-Ova cells, co-cultured with BMDCs to induce IFN-g in T-cells, indicating that the ability of both etoposide and mitoxantrone to induce immunogenicity in this model is RIPK1-dependent. In contrast, co-treatment of B16-Ova cells with Z-VAD only marginally reduced T-cell IFN-γ responses (by ˜12%) with etoposide and had no effect with mitoxantrone, indicating that the process was largely independent of caspases for both agents.

Furthermore, co-treatment of B16-Ova cells with the NF-kB signaling inhibitor Bay11-7085 and etoposide reduced the frequency of IFN-γ+ T-cells by >90% while co-treatment with Bay 11-7085 and mitoxantrone reduced the frequency of IFN-γ+ T-cells by >50% suggesting that NF-kB signaling in both etoposide and mitoxantrone-treated B16-Ova cells is important for the induction of DC-mediated T-cell IFN-γ responses. Finally, co-treatment of B16-Ova cells with the p38 MAPK inhibitor SB202190 and etoposide reduced the frequency of IFN-γ+ T-cells by ˜22% while co-treatment with SB202190 and mitoxantrone nearly abrogated the induction of IFN-γ+ T-cells altogether.

Consistent with these results, both etoposide and mitoxantrone, which induced DC-mediated T-cell priming, but not doxorubicin, which did not, were found to induce RIPK1 activation in B16-Ova cells when assayed by western blotting with an anti phoshoRIPK1(S166) antibody. Furthermore, western blotting of cell lysates with an anti-phospho-p38 antibody demonstrated p38MAPK activation by etoposide and mitoxantrone, as well as doxorubicin (which did not induce a DC-mediated T-cell IFN-γ response), indicating that induction of p38MAPK signaling in tumor cells is necessary but not sufficient for the induction of IFN-γ in T-cells. Taken together, these data indicate that active signaling through the RIPK1, NF-kB and p38MAPK signaling pathways in live but damaged tumor cells following chemotherapy treatment is needed for the induction of DC-mediated T-cell IFN-γ responses.

TABLE 7
Percentage of CD3+CD8+IFN-γ+ T cells
following B16-Ova cells co-treated with etoposide
or mitoxantrone in combination with necrostatin-1
or Z-VAD prior to co-culture with BMDC.

% CD3+CD8+IFN-γ+ T-cells

| | MEAN | SEM |
|---|---|---|
| Unstim | 0.02 | 0.00 |
| Etop 50 uM | 4.11 | 0.03 |
| Etop 50 uM + Z-vad | 5.70 | 0.05 |
| Etop 50 uM + Nec-1 | 1.04 | 0.13 |
| Mito 10 uM | 3.98 | 0.30 |
| Mito 10 uM + Z-vad | 3.65 | 0.26 |
| Mito 10 uM + Nec-1 | 0.27 | 0.04 |

### Example 4: In Situ Treatment of B16-Ova Tumors in Mice with Etoposide does not Synergize with Systemic Checkpoint Blockade

Materials and methods

Mouse Studies

B16-Ova cells or MC-38 cells (1×10⁶) were implanted subcutaneously in the right flank of 7-8 week old female C57BL/6J WT or BATF3 (−/−) mice. After 11-13 days tumors of −16 mm² median cross-sectional area were typically detectable by palpation. Mice with tumors were then binned into groups and injected intra-tumorally once a week for 3 weeks with 30 μl of either PBS, free etoposide to achieve a final concentration of 50 μM in the tumor volume, or 1×10⁶ etoposide-treated cells (24 hours of drug treatment followed by extensive washing with PBS). Where indicated, groups also received intra-peritoneal injections of 200 μg each of anti-PD1 (clone RMP1-14, BioXCell) and anti-CTLA4 (clone 9D9, BioXcell) twice a week for three weeks.

To enumerate circulating tumor antigen-specific CD8+ T-cells, mice were bled retro-orbitally after the second intra-tumoral dose of PBS, etoposide, or etoposide-treated tumor cells, and H2-Kb/SIINFEKL (SEQ ID NO:1)-tetramer positive CD8+ T-cells analyzed by flow cytometry. Briefly, 50 μl of whole blood was collected by retro-orbital bleeding, centrifuged at 250×g for 5 min, followed by 3 rounds of RBC lysis in 200 ul of ACK buffer. Cells were then washed once in Tetramer stain buffer (PBS containing 5 mM EDTA, 1% BSA and 50 nM Dasatinib), and stained with PE-conjugated Tetramer for 40 min at RT, followed by co-staining with anti-CD8 for 10 min at 4° C. Cells were then stained with DAPI, washed and re-suspended in tetramer stain buffer for flow cytometry analysis.

Tumor Size Measurements

Cross-sectional area of tumors was measured in mm² using calipers every 2-3 days. In tumor re-challenge experiments, naive mice controls or mice who had complete tumor regression and remained tumor free for at least 60 days were subcutaneously injected in the left flank (contra-lateral to the initial tumor) with 0.1×10⁶ B16-Ova cells, and tumor development was monitored for another 60 days.

Results

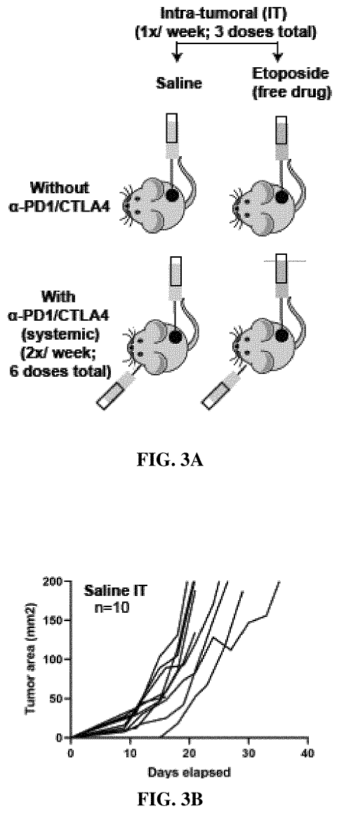

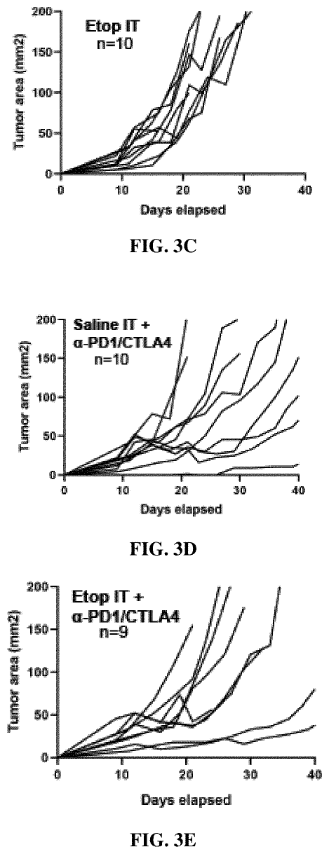

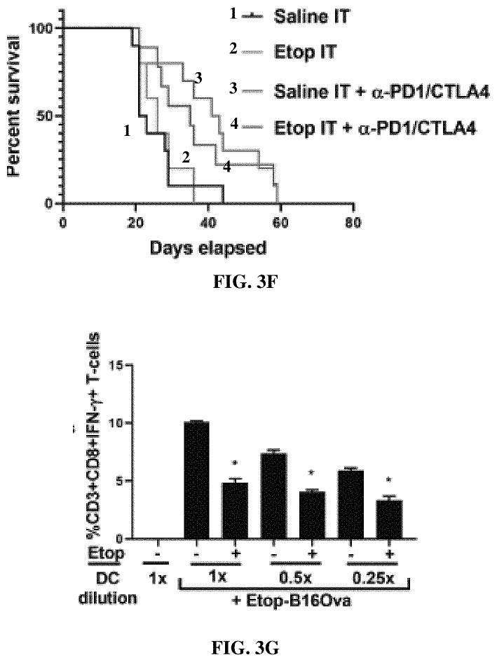

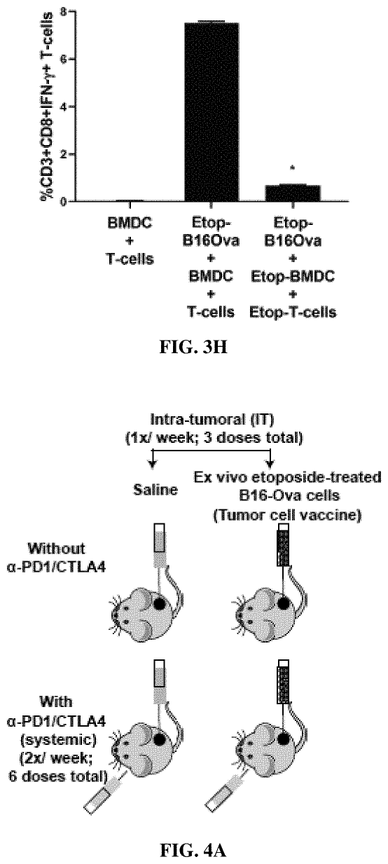

Given the ability of etoposide-treated B16-Ova cells to induce DC-mediated T-cell priming ex vivo, it was considered that intra-tumoral administration of etoposide could enhance DC function in vivo by increasing the immunogenicity of B16-Ova cells. This would be expected to induce antigen-specific T-cell expansion in vivo, particularly if used in combination with systemic immune checkpoint blockade. To test this, mice bearing flank B16-Ova tumors were treated by intra-tumoral administration of either saline or etoposide (three weekly doses) in the presence or absence of systemic anti-PD1 and anti-CTLA4 antibodies (two doses a week for three weeks) to confer immune checkpoint blockade (FIG. 3A). As shown in FIGS. 3B-3C, intratumoral injection of etoposide alone had no effect on tumor growth. Systemic administration of immune checkpoint blockade in combination with intra-tumoral chemotherapy also did not significantly enhance survival beyond that seen with immune checkpoint blockade alone (FIGS. 3D-3F). Furthermore, when the frequency of circulating H2-Kb/SIINFEKL (SEQ ID NO:1)-specific CD8+ T-cells was examined, no expansion of these cells when compared to the group that received checkpoint blockade alone was observed (not shown).

Intra-tumoral administration of etoposide, however, exposes both tumor cells and non-tumor cell types such as intra-tumoral DCs to this cytotoxic drug, which could potentially limit DC activation and impair the expansion of tumor antigen-specific T-cells. The assay shown in FIG. 1A was revised to now include co-exposure of both the BMDCs and tumor cells to etoposide prior to the addition of OT-1 T-cells. As shown in FIG. 3G, co-exposure of both BMDCs and tumor cells to etoposide significantly reduced the appearance of IFN-γ+ CD8+ T-cells compared to exposure of B16-Ova cells alone, indicating that exposure of DCs to etoposide impairs their ability to induce T-cell priming Consistent with this, the viability of BMDCs was significantly reduced upon exposure to etoposide. The assay was further modified to include exposure of all of the relevant cell types—tumor cells, BMDCs and T-cells—to etoposide, mirroring what might occur following intra-tumoral injection of the drug in vivo. This triple co-exposure resulted in an even more profound loss of tumor-directed T-cells to less than 10% of the level seen when etoposide exposure is limited to the tumor cells alone (FIG. 3H).

### Example 5: Intra-Tumoral Injection of Ex Vivo Etoposide-Treated Tumor Cells Synergizes with Immune Checkpoint Blockade, Enhances Survival and Induces Resistance to Re-Challenge

Materials and Methods

All assays were performed as described in the previous examples.

Results

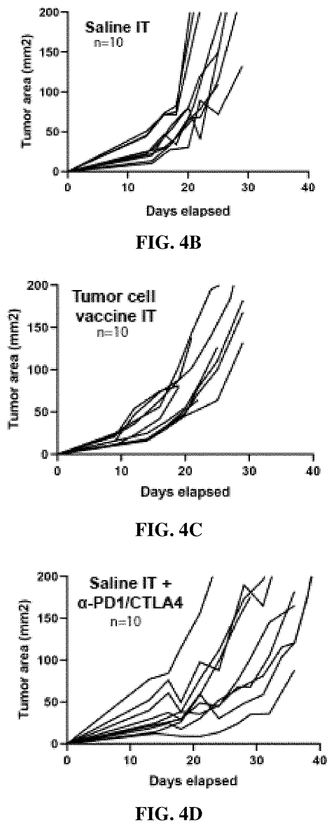

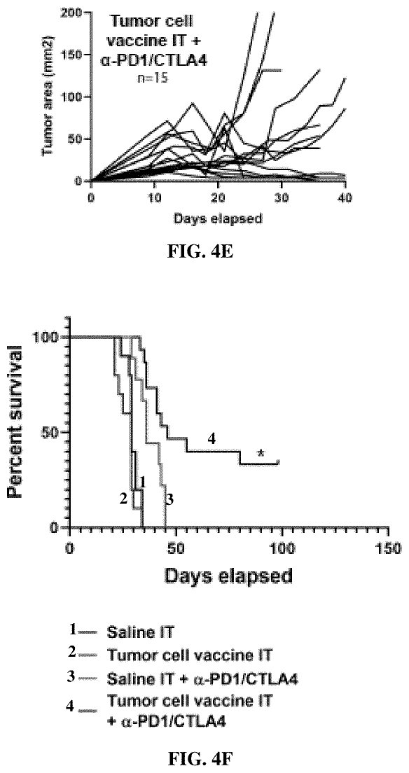

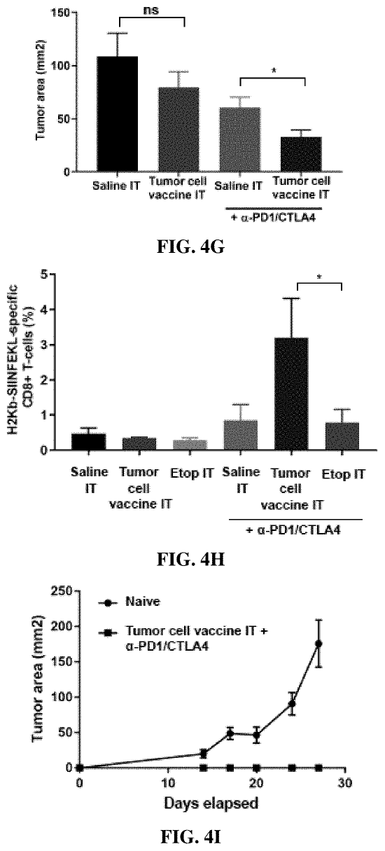

Exposure of BMDC and T-cells to etoposide reduced the induction of IFNg+CD8+ T-cells by drug-treated B16-Ova cells compared to etoposide exposure of B16-Ova cells alone. It was considered that the intra-tumoral injection of ex vivo etoposide-treated B16-Ova cells into B16-Ova tumors in vivo, rather than intra-tumoral injection of the free drug, would minimize exposure of other immune cell types in the tumor and draining lymph node to the cytotoxic effects of etoposide. To test this, mice bearing flank B16-Ova tumors received intra-tumoral injection of either saline or ex vivo etoposide-treated B16-Ova cells in the presence or absence of systemic checkpoint blockade (FIG. 4A). Intra-tumoral administration of ex vivo etoposide-treated tumor cells alone had no effect on subsequent tumor progression (FIGS. 4B-4C, 4F-4G). However, when used in combination with systemic checkpoint blockade, the mice displayed superior tumor control compared to those that received checkpoint blockade alone, resulting in complete tumor regressions in a subset of mice progression (FIGS. 4D-4E, 4G and Table 8). Furthermore, survival was also markedly enhanced in this group (FIG. 4F). Analysis of circulating lymphocytes in these animals revealed an enhanced frequency of H2-Kb/SIINFEKL (SEQ ID NO:1)-specific CD8+ T-cells (FIG. 4H and Table 9), indicating that intra-tumoral administration of ex vivo etoposide-treated tumor cells functions as an effective tumor cell vaccine, which in combination with immune checkpoint blockade, promotes efficient T-cell priming and anti-tumor immunity. The subset of mice that demonstrated complete tumor regression after tumor cell vaccine treatment remained tumor-free for at least 98 days (FIG. 4F). These complete responders and naive control mice (which were never previously exposed to B16-Ova tumor cells) were then re-challenged in the contralateral flank with live B16-Ova cells. FIG. 4I shows that tumors grew to 200 mm² cross-sectional area within 30 days in the naive mice, (at which point they were euthanized). Notably, none of the intra-tumoral vaccine-treated animals who were cured of their initial tumors after therapy developed tumors upon re-challenge, indicating that combining systemic checkpoint blockade with intra-tumoral injections of the tumor cell vaccine induces anti-tumor immunological memory.

TABLE 8
Tumor area (mm²) in mice treated with tumor cell vaccine and ICI.

Tumor area (mm²)

| | MEAN | SEM |
|---|---|---|
| Saline IT | 108.49 | 21.91 |
| Tumor cell vaccine IT | 79.22 | 14.92 |
| Saline IT + | 63.54 | 14.44 |
| α-PD1/CTLA4 | | |
| Tumor cell vaccine | 32.97 | 6.21 |
| IT + α-PD1/CTLA4 | | |

TABLE 9
Percentage of H2-Kb-SIINFEKL (SEQ ID NO: 1)-specific T-
cells in mice treated with tumor cell vaccine and ICI.

H2-Kb-SIINFEKL
(SEQ ID NO: 1)-specific T-cells (%)

| | MEAN | SEM |
|---|---|---|
| Saline IT | 0.47 | 0.15 |
| Tumor cell vaccine IT | 0.35 | 0.03 |
| Etop IT | 0.29 | 0.06 |
| Saline IT + | 0.86 | 0.44 |
| α-PD1/CTLA4 | | |
| Tumor cell vaccine | 3.20 | 1.11 |
| IT + α-PD1/CTLA4 | | |
| Etop IT + | 0.79 | 0.37 |
| α-PD1/CTLA4 | | |

To examine whether this response was unique to the B16 cell line, or to cells engineered to express the ovalbumin antigen, similar intra-tumoral injections of saline- or etoposide-treated tumor cells, were performed in the presence or absence of systemic immune checkpoint blockade, with MC-38 murine colon carcinoma cells that do not express ovalbumin. In this tumor model, there was minimal benefit of immune checkpoint blockade alone when the MC-38 tumors were injected with saline. Similarly, intra-tumoral injection of etoposide-treated MC-38 tumor cells into pre-existing MC-38 tumors failed to elicit an anti-tumor immune response in the absence of systemic immune checkpoint blockade. However, 20% of the animals who received the combination of the MC-38 tumor cell vaccine together with systemic immune checkpoint blockade showed complete tumor regression and prolonged survival.

### Example 6: Batf3 (−/−) Mice do not Respond to the Tumor Cell Vaccine and Checkpoint Blockade Combination

Materials and Methods

Immunophenotyping

Phenotypic characterization of immune cell populations was performed by flow cytometry. Briefly, tumors were harvested and mashed through a 70 μM filter. Collected cells were washed in FACS buffer (PBS containing 5 mM EDTA and 1% BSA), resuspended, and counted. Five million cells from each sample were stained with fluorophore-conjugated antibodies on ice for 30 min, co-stained with Aqua, washed, resuspended in 450 μl, supplemented with 50 μl of CountBright absolute counting beads, and analyzed on a BD LSR Fortessa flow cytometer. DCs were scored as CD45+Ly6CCD24+MHCII+F480−(CD11b+ or CD103+) cells using the gating strategy described in Broz M L, et al., Cancer Cell, 8; 26(6):938 (2014).

Results

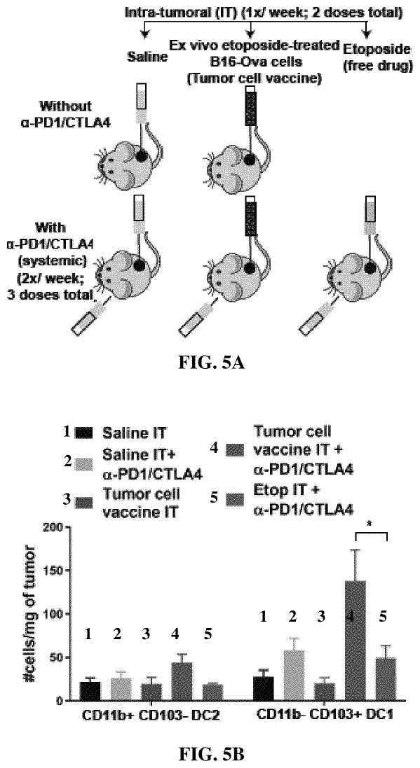

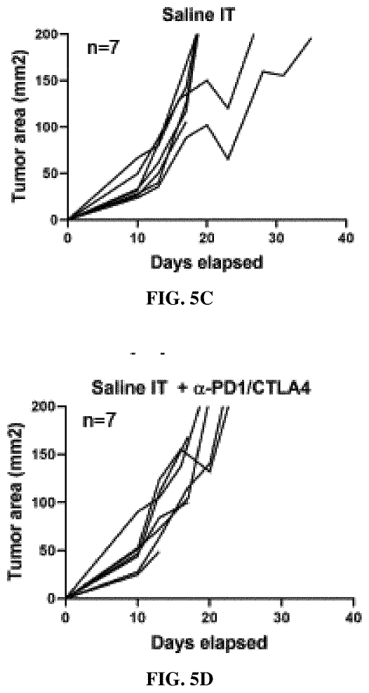

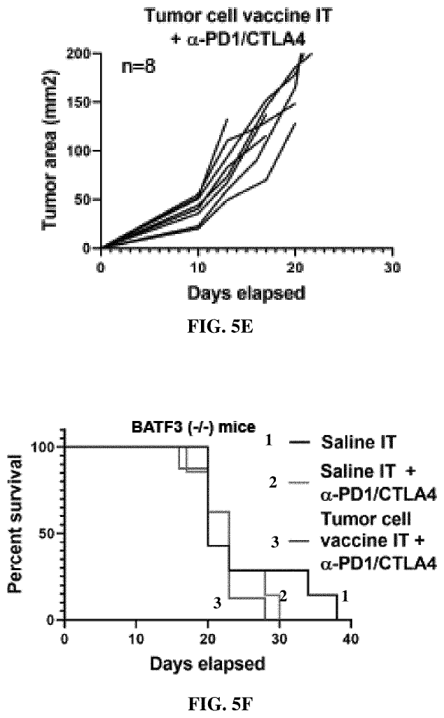

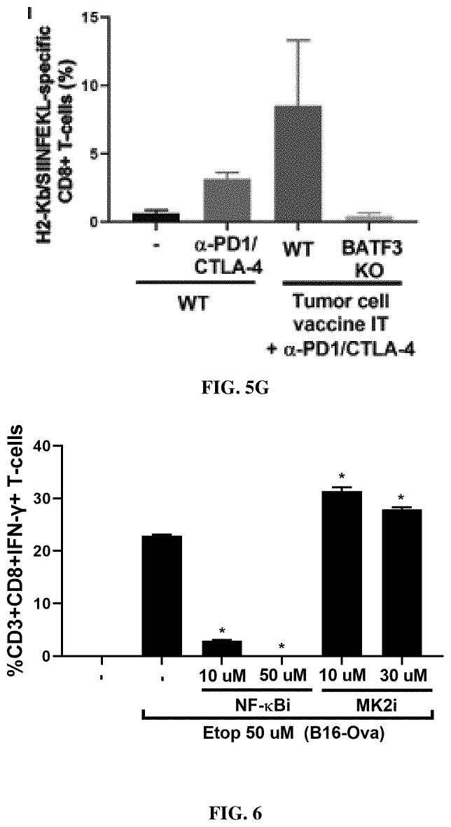

To test whether the efficacy of the tumor cell vaccine and checkpoint combination treatment for an anti-tumor immune response depends on DCs that can cross-present tumor antigens, the numbers of CD11b⁺C103⁻DC2 cells and CD11b⁻CD103⁺DC1 cells were enumerated by immunophenotyping and flow cytometry. CD11b⁻CD103⁺DC1 cells, which are typically also Batf3+(Edelson B T., et al., J Exp Med., 207(4):823-36 (2010); Merad M., et al., Annu Rev Immunol., 31:563-604 (2013)), are known to cross present tumor antigens to CD8+ T-cells (Hildner, Science., 322(5904):1097-100 (2008)). As before, mice bearing flank B16-Ova tumors were treated with saline or the tumor cell vaccine intra-tumorally, in the presence or absence of systemic checkpoint blockade (FIG. 5A), and analyzed. In addition, a cohort that was treated with intra-tumoral etoposide in combination with checkpoint blockade was also included. After 2 doses of the vaccine or etoposide and 3 doses of checkpoint blockade, immunophenotyping of the tumors revealed an enhanced number of CD103+DC1 in tumors that were being treated with tumor cell vaccine and checkpoint blockade, compared to the other groups (FIG. 5B). In addition, cross-sections of tumors treated with the tumor cell vaccine and checkpoint blockade showed markedly enhanced Batf3 staining by immunohistochemistry indicating the enhanced presence of Batf3+DC, which was not present in the other treatment groups. Intra-tumoral injection of free etoposide combined with checkpoint blockade did not enhance numbers of CD103+DC1, consistent with the lack of T-cell expansion and no enhancement in efficacy seen in vivo (FIG. 3A-3F) and in vitro (FIG. 3G-3H) with this treatment.

To directly validate the contribution of Batf3+CD11b-CD103+DC1 cells to antitumor immunity induced by the combination of the tumor cell vaccine and checkpoint blockade, the experiment shown in FIG. 4A was repeated using Batf3−/− mice. Intra-tumoral injection of ex vivo etoposide-treated tumor cells with systemic immune checkpoint blockade failed to induce tumor control or prolong the lifespan of tumor-bearing mice in the absence of Batf3 (FIG. 5C-5F). While the DNA damage-induced tumor cell vaccine and systemic ICI combination enhanced the frequency of circulating H2-Kb/SIINFEKL (SEQ ID NO:1)-reactive CD8+ T-cells in WT mice, there was no enhancement in Batf3-deficient mice (FIG. 5G). Taken together, these data strongly suggest that intra-tumoral administration of ex vivo etoposide-treated tumor cells as a tumor cell vaccine, in combination with systemic checkpoint blockade, promotes Batf3+DC-mediated anti-tumor T-cell responses leading to enhanced survival, and complete tumor regressions in a subset of mice concurrent with long-term anti-tumor immunological memory.

### Example 7: Enhancement of BMDC-Mediated T-Cell Priming with Etoposide and an MK2 Inhibitor

B16-Ova cells were co-treated with Etoposide and an NF-κB inhibitor (Bay 11-7085) or an MK2 inhibitor (PF-3644022). The B16-Ova cells were then co-incubated with BMDC cells, which were then used in the T cell priming assays.

It was observed that the co-treatment of B16-Ova cells with Etoposide and an NF-κB inhibitor (Bay 11-7085) inhibits BMDC-mediated T-cell priming while co-treatment with etoposide and an MK2 inhibitor (PF-3644022) enhances BMDC-mediated T-cell priming Quantification of IFN-γ+ CD8+ T-cells induced by BMDC following incubation with Etoposide-treated B16-Ova cells that were co-treated with either Bay 11-7085 (NF-κB inhibitor) or PF-3644022 (MK2 inhibitor) is shown in FIG. 6 and Table 10. The first lane (−) indicates the percentage of IFN-γ+ CD8+ T-cells produced by co-culture of BMDCs and T-cells in the absence of B16-Ova cells. Error bars indicate SEM. * indicates p<0.0001 when compared to cells treated with Etoposide (50 μM) alone using ANOVA followed by Dunnett's multiple comparisons test.

TABLE 10
Percentage of CD3+CD8+IFN-γ+ T-cells following B16-
Ova cells co-treated with etoposide or mitoxantrone in combination
with NF-κB inhibitor or MK2 inhibitor prior to co-culture with BMDC.

% CD3+CD8+IFN-γ+ T-cells

| | MEAN | SEM |
|---|---|---|
| | 0.01 | 0.01 |
| Etop 50 uM | 22.90 | 0.21 |
| Etop 50 uM + NF-Kbi 10 uM | 2.93 | 0.17 |
| Etop 50 uM + NF-Kbi 50 uM | 0.02 | 0.01 |
| Etop 50 uM + MK2i 10 uM | 31.40 | 0.70 |
| Etop 50 uM + MK2i 30uM | 27.93 | 0.38 |

These data showed that 1) NF-κB activation is required for enhancement of immunogenic potential of chemotherapy-treated tumor cells, and 2) co-treatment of tumor cells with Etoposide and an MK2 inhibitor further enhances immunogenic potential.

### Example 8: Live Injured Cells are More Efficient at Enhancing the Density of Intra-Tumoral Tumor-Antigen Specific CD8+ T-Cells than Dead Cells

Materials and Methods

For treatment of tumors, live injured cells and dead cells after etoposide treatment were generated as described in Example 2 above, under “Fractionation of live and dead fractions from chemotherapy-treated cells”.

Phenotypic characterization of T-cells from tumors was performed by flow cytometry. Briefly, tumors were excised, weighed and mashed through a 70 μM filter. Collected cells were washed in FACS buffer (PBS containing 5 mM EDTA and 1% BSA) and resuspended at 20 mg of tumor per 100 ul. Cells were stained with fluorophore-conjugated antibodies on ice for 30 min, co-stained with Aqua, washed, resuspended in 200 μl, supplemented with 25 μl of CountBright absolute counting beads, and analyzed on a BD LSR Fortessa flow cytometer. SIINFEKL (SEQ ID NO:1)-specific T-cells were scored as CD45+CD3+CD8+(H2-Kb-SIINFEKL (SEQ ID NO:1)-Tetramer)+cells.

Results

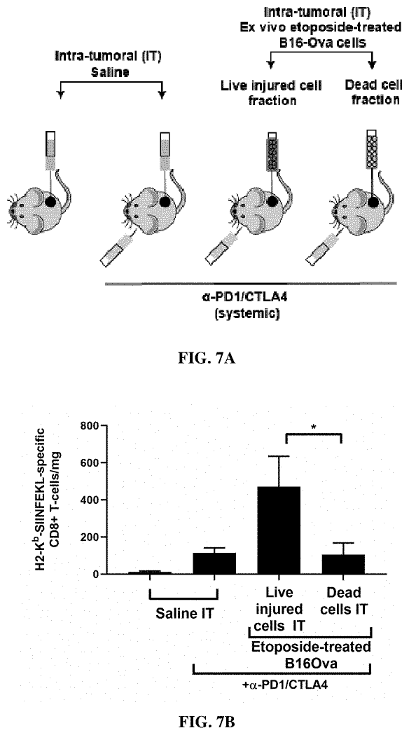

An experiment was designed to compare tumor infiltration of SIINFEKL (SEQ ID NO:1)-specific T-cells induced by the live injured cell fraction versus the dead cell fraction from the etoposide-treated B16-Ova cell mixture. An illustration of the experimental protocol is in FIG. 7A.

The results are illustrated in FIG. 7B, which shows quantification of H2-Kb-SIINFEKL (SEQ ID NO:1)-specific CD8+ T-cells per mg of tumor in the groups in indicated.

Results show that intra-tumoral administration of the live injured B16-Ova cell fraction after etoposide treatment is more efficient at enhancing the density of intra-tumoral tumor-antigen specific CD8+ T-cells compared to the dead cell fraction.

### Example 9: Inhibition of Specific DNA-Damage Signaling Pathways in Etoposide-Treated B16-Ova Cells Impairs Dendritic-Cell Mediated T-Cell Activation

Materials and Methods

Live injured cell fraction was generated as described in Example 2 above, under “Fractionation of live and dead fractions from chemotherapy-treated cells”.

The assay was performed as described in Example 1 above, under “In vitro cross presentation assay”.

Results

An experiment was designed to determine if DNA-damage signaling pathways influence etoposide-treated cell activation of T-cells.

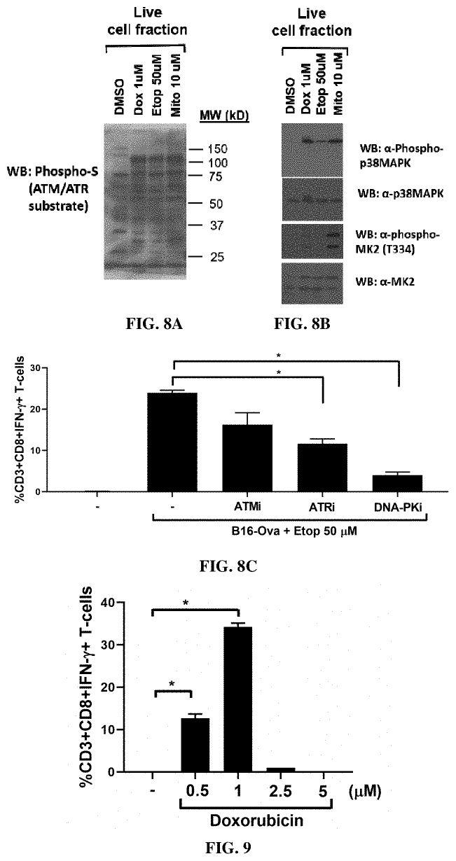

The live cell fractions from specific chemotherapy-treated B16-Ova cell mixtures were analyzed by western blotting for serine-phosphorylated substrates of ATM and ATR (FIG. 8A) and also for phospho- and total p38MAPK as well as phospho (T334)- and total MK2 (FIG. 8B).

FIG. 8C shows quantification of IFN-γ+ CD8+ T-cells induced by BMDC following incubation with etoposide-treated B16-Ova cells that were co-treated with either KU-55933 (ATM inhibitor), AZD6738 (ATR inhibitor) or NU7441 (DNA-PK inhibitor). The first lane (−) indicates the percentage of IFN-γ+ CD8+ T-cells produced by co-culture of BMDCs and T-cells in the absence of B16-Ova cells. Error bars indicate SEM. * indicates p<0.0001 when compared to cells treated with Etoposide (50 uM) alone using ANOVA followed by Dunnett's multiple comparisons test.

The results indicate that inhibition of specific DNA-damage signaling pathways in etoposide-treated B16-Ova cells impairs dendritic-cell mediated T-cell activation.

### Example 10: B16-Ova Cells Treated with Specific Doses of Doxorubicin, when Co-Cultured with BMDC, Promote IFN-Gamma Production in CD8+ T-Cells

Materials and Methods

The assay was performed as described in Example 1 above, under “In vitro cross presentation assay”.

Results

An experiment was designed to test the ability doxorubicin-treated cells to induce IFN-gamma production in CD8+ T-cells

Results are illustrated in FIG. 9, which shows quantification of IFN-γ+ CD8+ T-cells induced by BMDC following incubation with doxorubicin-treated B16-Ova cells at the doses indicated. The first lane (−) indicates the percentage of IFN-γ+ CD8+ T-cells produced by co-culture of BMDCs and T-cells in the absence of B16-Ova cells. Error bars indicate SEM. * indicates p<0.0001 when compared to cells treated with (−) using ANOVA followed by Dunnett's multiple comparisons test.

The results shows that B16-Ova cells treated with specific doses of doxorubicin, when co-cultured with BMDC, promote IFN-gamma production in CD8+ T-cells.

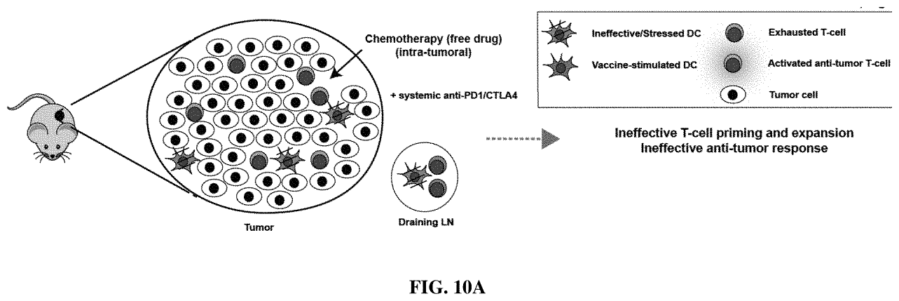

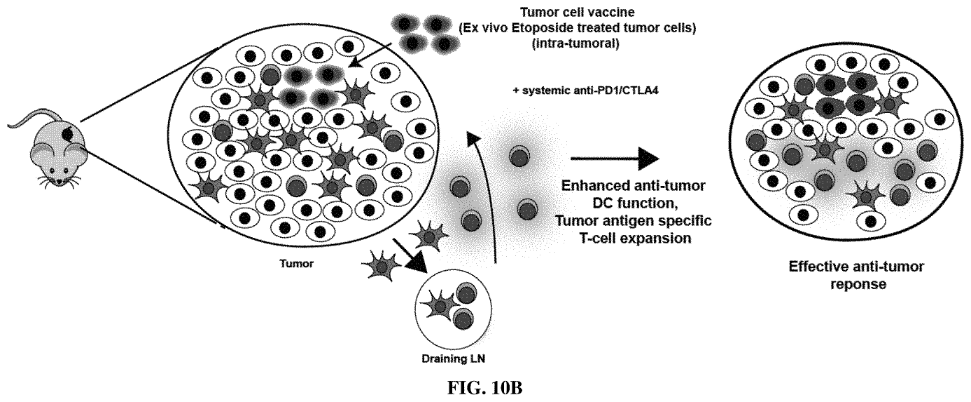

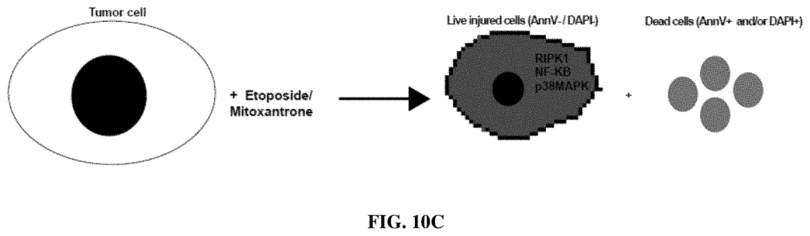

Presented here is one specific modality combining chemotherapy with ICI. The synergy between these two treatment methods was accomplished by creating a vaccine from ex vivo chemotherapy-treated tumor cells (FIG. 10B). To do this, in vitro immunogenicity assay was used to identify specific doses of etoposide and mitoxantrone that, when used to treat B16-Ova cells, effectively induced DC-mediated T-cell priming. When this approach was translated in vivo by direct intra-tumoral injection of etoposide, in combination with systemic ICI administration, however, the therapy was largely ineffective. The exposure of DCs or T-cells to etoposide dramatically impaired T-cell priming. An altered therapeutic approach was used instead by performing an intratumoral injection of ex vivo etoposide treated B16-Ova cells directly into existing B16-Ova tumors. When this was combined with systemic administration of ICI, an expansion of CD103+ intra-tumoral DCs, an increase in the frequency of H2-K^{b}/SIINFEKL (SEQ ID NO:1)-reactive circulating anti-tumor CD8+ T-cells were observed, and markedly enhanced tumor control and significant survival benefit was achieved compared to ICI alone. Furthermore, a subset of mice showed complete tumor regressions and resistance to re-challenge with live tumor cells in the contra-lateral flank. A similar response was observed using MC-38 cells lacking ovalbumin, indicating that the results were not limited to one tumor cell type, or to cells that express a foreign non-tumor antigen.

The finding that certain types of DNA-damaging chemotherapy could increase the immunogenicity of the treated tumor cells is in good agreement with many findings from Obeid et al., Nat Med., 13(1):54-61 (2007). The immunogenicity assay used by Obeid et al differs substantially from the assay used here. In their system, drug-treated tumor cells were injected into the flank of naive mice, and the mice then challenged with undamaged tumor cells injected into the opposite flank 7 days later. Failure of the second tumor cell challenge to establish a tumor was taken as evidence of anti-tumor immunity. Here, the ability of drug-treated cells to drive the priming of CD8+ T-cells for IFN-g production was directly measured, and this effect was further validated in vivo for etoposide treatment by injection of the drug-treated tumor cells into pre-existing mouse tumors, followed by direct measurements of tumor response and tumor-infiltrating immune cells in the presence or absence of systemic immune checkpoint inhibitors.

Knock-down of calreticulin prior to etoposide exposure only partially reduced the ability of these cells to induce DC-dependent T-cell priming, which could also not be explained by drug-induced HMGB1 or ATP release. Together with the finding that the dead cells or cell-free supernatants alone, or in combination, when co-incubated with BMDC, were not sufficient to induce IFN-γ in T-cells and that active signaling in the live injured fraction of cells after etoposide or mitoxantrone treatment is important for DC-mediated T-cell IFN-γ responses raises several interesting possibilities about the mechanisms involved in promoting effective cross-presentation of tumor antigens by DCs to T-cells. Current understanding presumes that a property of dead cells generated by chemotherapy, such as specific molecules presented on the cell surface or released into the microenvironment, are the major determinants of effective cross-presentation of tumor antigens by DC to T-cells. The findings discussed herein instead indicate that active signaling through RIPK1, NF-kB and p38MAPK by live but stressed and injured cells after chemotherapy treatment are a major determinant of efficient DC-mediated T-cell priming. However, the results do not exclude a contribution from chemotherapy-induced cell death, since some of the live injured cells after chemotherapy treatment may die during the co-incubation period with BMDCs. Finally, lysates of the chemotherapy-treated cell mixture generated by three cycles of freeze-thawing, when co-incubated with DC, do not promote T-cell IFN-γ response indicating that an active cellular process beyond cytokine secretion may be involved.

The finding that RIPK1 and NF-kB are involved in driving immunogenic cell death following treatment of tumor cells with specific DNA damaging chemotherapeutic drugs is in excellent agreement with the recent results of Yatim et al., (Yatim et al., Science, 350(6258):328-334 (2015)) and Snyder et al., (Snyder et al., Sci. Immunol. 4, eaaw2004 (2019)).

Tumor cell vaccines have been in various stages of development for almost three decades, but have yet to show robust clinical efficacy in large unselected cancer patient populations (Dranoff et al., Proc Natl Acad Sci U S A., 90(8):3539-43 (1993), Lipson et al., J Transl Med., 13:214 (2015)). The best prototype to date, GVAX, consists of irradiated cancer cells engineered to secrete GM-CSF, and is well tolerated in patients, however, it has not been successful in clinical trials so far. Notably, these vaccines are administered intradermally, rather than directly into the tumor, and therefore do not directly access the stimulatory CD103+ DCs in the tumor microenvironment. Gaining access to intra-tumoral and/or tumor-draining lymph node DC may be crucial in re-activating the DC-T-cell axis of antitumor immunity. Furthermore, GVAX and other contemporary tumor cell vaccines are not specifically enhanced for immunogenicity using in vitro assays of T-cell priming with patient-matched immune cells.

Described is also a therapeutic method without requiring the need to genetically manipulate the cells to artificially drive RIPK3 dimerization (Yatim et al., Science, 350(6258):328-334 (2015); Snyder et al., Sci. Immunol. 4, eaaw2004 (2019)). The method includes tumor cells derived from patient tumor biopsies, expanded and used to screen the immunogenicity of chemotherapeutic compounds to identify the optimal compound for a particular tumor using primary patient-derived or allogeneic DC and CD8+ T-cells. Matched tumor cells treated with the optimal compound identified are then be re-injected into the same tumor in combination with systemic checkpoint blockade. This approach may be useful for patients whose cancers are accessible for intra-tumoral delivery and in whom conventional treatment options have failed and initial or acquired resistance to ICI has been observed.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A composition for treating a patient with cancer, and/or preventing recurrence of the cancer, the composition comprising isolated, activated, primary tumor cells.

2. The composition of claim 1, wherein the cells are live, injured cells.

3. The composition of claim 2, wherein the isolated activated cells are activated with one or more genotoxic drugs selected from the group consisting of alkylating agents, antimetabolites, antimitotics, anthracyclines, cytotoxic antibiotics, and topoisomerase inhibitors, and, optionally, with one or more MAPK-activated protein kinase-2 (MK2) inhibitors.

4. The composition of claim 3, wherein the concentration of drug is sufficient to injure the cells and induce stress signaling, but not sufficient to induce maximal cell death of the cells.

5. The composition of claim 4, wherein the genotoxic drug is selected from the group consisting of doxorubucin, etoposide, mitoxantrone, cisplatin, oxaliplatin, 5-fluorouracil, paclitaxel, irinotecan, camptothecin, and cyclophosphamide.

6. The composition of claim 4, wherein the isolated activated tumor cells comprise cells with DNA damage, growth arrest, and/or necroptosis.

7. The composition of claim 4, wherein the cells comprise induced or increased phosphorylation of p38MAPK and/or intact, induced, or increased DNA damage signaling, optionally wherein the DNA damage signaling comprises phosphorylation of one or more substrates of protein kinase ataxia-telangiectasia mutated (ATM), serine/threonine-protein kinase ATR, or a combination thereof.

8. The composition of claim 1, wherein the cells are free from in vitro or ex vivo transformation or transfection of a heterologous nucleic acid expression construct.

9. The composition of claim 1, wherein the cells are in vitro or ex vivo transformed or transfected with a heterologous nucleic acid expression construct for expression of one or more cytokines and/or signaling molecules, preferably wherein the cytokines and/or signaling molecules are downstream of RIPK1 and NF-kB, optionally wherein at least one of the cytokines is GM-CSF.

10. The composition of claim 1, further comprising dendritic cells, and/or T cells.

11. The composition of claim 1, further comprising one or more immune checkpoint inhibitors (ICI), optionally wherein the ICI is a small molecule, antibody, or antibody fragment against a molecule selected from the group consisting of programmed cell death protein 1 (PD-1), PD-1 Ligand 1 (PD-L1), and cytotoxic T-lymphocyte-associated antigen 4 (CTLA-4).

12. A method of treating a patient with cancer, and/or preventing recurrence of the cancer, comprising administering to the patient an effective amount of the composition of claim 1.

13. The method of claim 12, wherein the composition is administered by intratumoral injection.

14. The method of claim 13, comprising administering to the patient an effective amount of one or more immune checkpoint inhibitor(s) (ICI).

15. The method of claim 33, wherein the ICI is administered before, during, or after administering the composition.

16. The method of claim 12, wherein the composition comprises between about 10⁴ and about 10⁹ isolated activated tumor cells activated with an effective amount of one or more genotoxic drug(s), optionally treated with one or more MAPK-activated protein kinase-2 (MK2) inhibitors.

17. The method of claim 16, wherein the composition comprises tumor cells isolated from a tumor of the patient.

18. An ex vivo assay for personalized treatment of a patient with cancer, the assay comprising:
treating a plurality of samples of tumor cells isolated from the patient with genotoxic drugs to produce activated cells, and
selecting a drug and/or dosage or concentration thereof that produces activated tumor cells with the increased immunogenic potential as the drug for the personalized treatment of the patient with cancer, optionally wherein the drug produces activated tumor cells with the highest immunogenic potential of the tested drugs.

19. The assay of claim 18, wherein each sample of the isolated tumor cells is treated with a single genotoxic drug.

20. The assay of claim 19, wherein the genotoxic drug is at a concentration between about 0.1 μM and about 1000 μM.

21. The assay of claim 20, wherein the cells are contacted with different amounts of the genotoxic drug to identify a dosage or concentration that injures the cells and induces stress signaling, but is not sufficient to induce maximal cell death of the cells.

22. The assay of claim 21, wherein the stress signaling comprises a DNA damage signaling pathway.

23. The assay of claim 18, wherein identifying is by
(i) detecting at least 1% necroptosis in the activated tumor cells, as measured by flow cytometry,
(ii) detecting activated receptor-interacting protein kinase 1 (RIPK1), NF-κB, or combination thereof in the activated tumor cells, optionally as measured by Western blotting and/or flow cytometry, or
(iii) a combination thereof.

24. The assay of claim 18, wherein the assay further comprises co-culturing the produced activated cells with patient's dendritic cells.

25. The assay of claim 24, wherein the assay further comprises co-culturing the produced activated cells with patient's T cells.

26. The assay of claim 18, comprising testing the produced activated tumor cells for improved dendritic-cell mediated T-cell priming.

27. A personalized treatment of a patient with cancer, comprising administering into a tumor of the patient an effective amount of the patient's own activated tumor cells having an increased immunogenic potential, and optionally the highest immunogenic potential, as prepared according to the assay of claim 18.

28. The personalized treatment of claim 51, wherein the effective amount of the patient's own activated tumor cells comprises an amount between about 10⁴ and about 10⁹ cells activated tumor cells.

29. The personalized treatment of claim 27, further comprising administering the patient an effective amount of one or more immune checkpoint inhibitors (ICI).

30. The personalized treatment of any one of claims 51-55, wherein the ICI is a small molecule or antibody or antibody fragment against a molecule selected from the group consisting of programmed cell death protein 1 (PD-1), against PD-1 Ligand 1 (PD-L1), and against cytotoxic T-lymphocyte-associated antigen 4 (CTLA-4).


SEQUENCE LISTING (1 sequence)

SEQ ID NO: 1 - protein, 8 aa, Artificial Sequence
  synthetic polypeptide
SIINFEKL